(19) United States
(12) Patent Application Publication
(10) Pub. No.: US 2017/0260134 A1
(43) Pub. Date: Sep. 14, 2017

(54) Ceramide Analogs

(71) Applicant: Augusta University Research Institute, Inc., Augusta, GA (US)

(72) Inventors: Kebin Liu, Martinez, GA (US); Iryna Lebedyeva, Augusta, GA (US); Genevieve Coe, Washington, GA (US)

(21) Appl. No.: 15/455,665

(22) Filed: Mar. 10, 2017

Related U.S. Application Data
(60) Provisional application No. 62/307,540, filed on Mar. 13, 2016.
(60) Provisional application No. 62/310,522, filed on Mar. 18, 2016.

(51) Int. Cl. C07D 209/42

(52) U.S. Cl. CPC C07D 209/42

(57) ABSTRACT

Ceramide analogs and methods of their use are provided.

### CROSS-REFERENCE TO RELATED APPLICATIONS

This application claims benefit of and priority to U.S. Provisional Patent Application Nos. 62/307,540 filed on Mar. 13, 2016, and 62/310,522 filed on Mar. 18, 2016, both of which are incorporated by reference in their entireties.

### STATEMENT REGARDING FEDERAL SPONSORED RESEARCH

This invention was made with government support under CA182518 awarded by the National Institutes of Health and under 1I01BX001962 awarded by the Department of Veterans Affairs. The government has certain rights in the invention.

### FIELD OF THE INVENTION

The invention is generally directed to ceramide analogs and their use to promote cancer cell sensitivity to immune response.

### BACKGROUND OF THE INVENTION

Fas, also termed CD95, APO1 or TNFRSF6, is a member of the tumor necrosis factor receptor superfamily. Fas exists as a trimeric membrane-bound surface receptor and is expressed on almost all types of cells throughout the mammalian body (Kaufmann, T., Strasser, A. & Jost, P. J. Fas death receptor signalling: roles of Bid and XIAP. Cell Death Differ 19, 42-50 (2012)). In contrast, the expression of the physiological ligand of Fas, Fas ligand (FasL, CD95L, or TNFSF6), is restricted to highly selective types of cells, primarily to activated T cells, NKT cells and NK cells (Aggarwal, B. B. Signalling pathways of the TNF superfamily: a double-edged sword. Nat Rev Immunol 3, 745-756 (2003) and Nagata, S. Fas ligand-induced apoptosis. Annu Rev Genet 33, 29-55, (1999)). Expression of FasL on certain non-lymphoid tissues, such as the eye and testis, has been reported but both its expression and function are still controversial (Allison, J., et al., Transgenic expression of CD95 ligand on islet beta cells induces a granulocytic infiltration but does not confer immune privilege upon islet allografts. Proc Natl Acad Sci USA 94, 3943-3947 (1997)). FasL has also been reported to be expressed in certain tumor cells, mainly as soluble FasL (Song, E. et al., Soluble Fas ligand released by colon adenocarcinoma cells induces host lymphocyte apoptosis: an active mode of immune evasion in colon cancer. Br J Cancer 85, 1047-1054 (2001); O'Connell, et al., The Fas counterattack: Fas-mediated T cell killing by colon cancer cells expressing Fas ligand. J Exp Med 184, 1075-1082 (1996) and O'Callaghan, G. et al. Targeting the EP1 receptor reduces Fas ligand expression and increases the antitumor immune response in an in vivo model of colon cancer. Int J Cancer 133, 825-834 (2013)). The expression and function of soluble FasL in tumor cells are hotly debated (Houston, A. M. et al. The “Fas counterattack” is not an active mode of tumor immune evasion in colorectal cancer with high-level microsatellite instability. Hum Pathol 39, 243-250 (2008)). However, it is generally believed that only the membrane-bound form of FasL is capable of inducing apoptosis (LA, O. et al. Membrane-bound Fas ligand only is essential for Fas-induced apoptosis. Nature 461, 659-663 (2009)).

Engagement of Fas receptor by soluble FasL has been shown to initiate non-apoptotic survival signals (Chen, L. et al. CD95 promotes tumor growth. Nature 465, 492-496 (2010); Peter, M. E. et al. The CD95 receptor: apoptosis revisited. Cell 129, 447-450 (2007); and Li, H. et al. Fas Ag-FasL coupling leads to ERK1/2-mediated proliferation of gastric mucosal cells. Am J Physiol Gastrointest Liver Physiol 294, G263-275 (2008)). However, the first and best-characterized function of Fas is its ability to mediate apoptosis in various types of cells, ranging from the so called type 1 lymphocytes to type 2 hepatocytes and epithelial tumor cells (Kaufmann, T., Strasser, A. & Jost, P. J. Fas death receptor signalling: roles of Bid and XIAP. Cell Death Differ 19, 42-50 (2012); Krammer, P. H. CD95 (APO-1/Fas)-mediated apoptosis: live and let die. Adv. Immunol. 71, 163-210 (1999); Wang, Y., et. al., Novel mechanism of harmaline on inducing G2/M cell cycle arrest and apoptosis by up-regulating Fas/FasL in SGC-7901 cells. Sci Rep 5, 18613 (2015); and Wang, S. et al. FAS rs2234767 and rs1800682 polymorphisms jointly contributed to risk of colorectal cancer by affecting SP1/STAT1 complex recruitment to chromatin. Sci Rep 6, 19229, (2016)). Binding of Fas by membrane-bound FasL of activated T cells, recombinant hexameric form FasL or Fas agonist mAb initiates the cell death cascade. Fas monomers first aggregate into trimers on the cell membrane of target cells, resulting in conformational changes in Fas receptor and resultant formation of the death-inducing signaling complex (DISC) of its cytoplasmic domain. DISC formation includes recruitment of the adaptor protein FADD through a homotypic interaction between the Fas cytoplasmic death domains and the C-terminus of FADD. Procaspase 8 is then recruited into the DISC through its N-terminal death-effector domain association with the N-terminal death-effector domain of FADD. Procaspase-8 is an inactive aspartate-specific cysteine protease. However, once recruited into the DISC, procaspase-8 acquires enzymatic activity through proximity-driven conformational changes, leading to the auto-proteolytic processing into active subunits which assemble into hetero-tetrameric enzymes. The activated caspase-8 then departs the DISC to target its specific substrates within the cytosol, such as effector caspase-3 and caspase 7, to initiate apoptosis (Kaufmann, T., Strasser, A. & Jost, P. J. Fas death receptor signalling: roles of Bid and XIAP. Cell Death Differ 19, 42-50 (2012); Nagata, S. Fas ligand-induced apoptosis. Annu Rev Genet 33, 29-55 (1999); Jost, P. J. et al. XIAP discriminates between type I and type II FAS-induced apoptosis. Nature 460, 1035-1039 (2009); and Chang, B. J. et al. Identification of the Calmodulin-Binding Domains of Fas Death Receptor. PLoS One 11, e0146493, (2016)).

In type 2 cells, such as hepatocytes and epithelial tumor cells, amplification of the caspase cascade is critical for Fas-mediated apoptosis (Jost, P. J. et al. XIAP discriminates between type I and type II FAS-induced apoptosis. Nature 460, 1035-1039 (2009)). Activated caspase-8 can cleave Bid into tBid. Once cleaved, tBid translocates to the mitochondrial outer membrane, resulting in activation of Bax/Bak. Bax/Bak mediate the mitochondrial outer membrane permeabilisation to release apoptogenic factors, including cytochrome c and the IAP antagonist Smac/DIABLO, from mitochondria into the cytosol, resulting in activation of the caspase activation cascade and apoptosis through Apaf-1-mediated activation of the initiator caspase-9 and downstream effector caspases. Therefore, caspase-8 bridges the crosstalk between the extrinsic apoptosis and the Bcl-2-regulated intrinsic apoptotic pathways within the Fas-mediated apoptosis pathway (Jost, P. J. et al. XIAP discriminates between type I and type II FAS-induced apoptosis. Nature 460, 1035-1039 (2009); and Gajate, C., Gonzalez-Camacho, F. & Mollinedo, F. Lipid raft connection between extrinsic and intrinsic apoptotic pathways. Biochem Biophys Res Commun 380, 780-784 (2009)).

Fas is highly expressed in normal human colon epithelial cells. It has been shown that Fas protein level is down-regulated in primary human colon carcinoma and complete loss of Fas expression often occurs in metastatic human colon carcinoma (Moller, P. et al. Expression of APO-1 (CD95), a member of the NGF/TNF receptor superfamily, in normal and neoplastic colon epithelium. Int J Cancer 57, 371-377 (1994)). It is known that FasL of cytotoxic T lymphocytes (CTLs) plays an essential role in suppression of spontaneous tumor development Afshar-Sterle, S. et al. Fas ligand-mediated immune surveillance by T cells is essential for the control of spontaneous B cell lymphomas. Nat Med 20, 283-290 (2014); Caldwell, S. A., et al., The Fas/Fas ligand pathway is important for optimal tumor regression in a mouse model of CTL adoptive immunotherapy of experimental CMS4 lung metastases. J Immunol 171, 2402-2412 (2003)); and Fingleton, B., Carter, K. J. & Matrisian, L. M. Loss of functional Fas ligand enhances intestinal tumorigenesis in the Min mouse model. Cancer Res 67, 4800-4806 (2007)). Therefore, human colon carcinoma may use down-regulation of Fas expression as a mechanism to escape host cancer immune surveillance. Therefore, therapeutic means to upregulate Fas expression level may be an effective way to suppress human colon carcinoma immune evasion. Because Fas receptor clustering and oligomerization is essential for Fas function (Cremesti, A. et al. Ceramide enables fas to cap and kill. J Biol Chem 276, 23954-23961 (2001); Sanchez, M. F., Levi, V, Weidemann, T. & Carrer, D. C. Agonist mobility on supported lipid bilayers affects Fas mediated death response. FEBS Lett 589, 3527-3533 (2015); Gajate, C., Gonzalez-Camacho, F. & Mollinedo, F. Involvement of raft aggregates enriched in Fas/CD95 death-inducing signaling complex in the antileukemic action of edelfosine in Jurkat cells. PLoS One 4, e5044 (2009); and Stel, A. J. et al. Fas receptor clustering and involvement of the death receptor pathway in rituximab-mediated apoptosis with concomitant sensitization of lymphoma B cells to fas-induced apoptosis. J Immunol 178, 2287-2295 (2007)), alternatively, therapeutic means to enhance Fas activation and resultant caspase-8 activation may represent another effective approach to suppress human colon carcinoma immune escape.

Ceramide, the central metabolite of the sphingolipid metabolism pathway, is a key secondary messenger that mediates multiple cellular functions, including cell proliferation, apoptosis, motility, differentiation, stress responses, protein synthesis, carbohydrate metabolism, immunity, and angiogenesis (Ogretmen, B. & Hannun, Y. A. Biologically active sphingolipids in cancer pathogenesis and treatment. Nat Rev Cancer 4, 604-616 (2004)). Compelling experimental data from mouse models and human patients have shown that ceramide deregulation is a key factor in tumor progression and cancer cell resistance to chemotherapeutic agents and radiation (Apraiz, A. et al. Evaluation of bioactive sphingolipids in 4-HPR-resistant leukemia cells. BMC Cancer 11, 477 (2011); and Cheng, J. C. et al. Radiation-induced acid ceramidase confers prostate cancer resistance and tumor relapse. J Clin Invest 123, 4344-4358 (2013)). The crucial roles of ceramide in tumor development and cancer cell responses to chemotherapy and radiation have led to extensive studies to target the ceramide metabolism pathways for development of potential anticancer therapies. For the last two decades, extensive efforts have been devoted to develop ceramide analogs to mimic natural ceramide, and numerous ceramide analogs with different chemical and biological properties (Singh, A., Ha, H. J., Park, J., Kim, J. H. & Lee, W. K. 3,4-Disubstituted oxazolidin-2-ones as constrained ceramide analogs with anticancer activities. Bioorg Med Chem 19, 6174-6181 (2011); Niiro, H. et al. (3Z)-2-Acetylamino-3-octadecen-1-ol as a potent apoptotic agent against HL-60 cells. Bioorg Med Chem 12, 45-51 (2004); Gududuru, V. et al. Synthesis and biological evaluation of novel cytotoxic phospholipids for prostate cancer. Bioorg Med Chem Lett 14, 4919-4923, (2004); Bieberich, E., Kawaguchi, T. & Yu, R. K. N-acylated serinol is a novel ceramide mimic inducing apoptosis in neuroblastoma cells. J Biol Chem 275, 177-181 (2000); Kim, K. et al. Synthesis and cytotoxicity of new aromatic ceramide analogs with alkylsulfonamido chains. Arch Pharm Res 30, 570-580 (2007); Antoon, J. W. et al. Design, synthesis, and biological activity of a family of novel ceramide analogues in chemoresistant breast cancer cells. J Med Chem 52, 5748-5752 (2009); Liu, J., Antoon, J. W., Ponnapakkam, A., Beckman, B. S. & Foroozesh, M. Novel anti-viability ceramide analogs: design, synthesis, and structure-activity relationship studies of substituted (S)-2-(benzylideneamino)-3-hydroxy-N-tetradecylpropanamides. Bioorg Med Chem 18, 5316-5322 (2010); Antoon, J. W. & Beckman, B. S. Anti-proliferative effects of the novel ceramide analog (S)-2-(benzylideneamino)-3-hydroxy-N-tetrade-cylpropanamide in chemoresistant cancer. Bioorg Med Chem Lett 22, 2624-2628 (2012); Bielawska, A. et al. Novel analogs of D-e-MAPP and B13. Part 2: signature effects on bioactive sphingolipids. Bioorg Med Chem 16, 1032-1045 (2008); and Selzner, M. et al. Induction of apoptotic cell death and prevention of tumor growth by ceramide analogues in metastatic human colon cancer. Cancer Res 61, 1233-1240 (2001)). However, these ceramide analogs are primarily designed for its direct anti-cancer activity.

Although trimerized Fas can initiate apoptosis, it seems that super-aggregation of trimerized Fas may enhance FasL-induced apoptosis via a ceramide-dependent mechanism in both type 1 and type 2 cells (Cremesti, A. et al. Ceramide enables fas to cap and kill. J Biol Chem 276, 23954-23961 (2001); Park, M. A. et al. Vorinostat and sorafenib increase CD95 activation in gastrointestinal tumor cells through a Ca(²⁺)-de novo ceramide-PP2A-reactive oxygen species-dependent signaling pathway. Cancer Res 70, 6313-6324 (2010); Zhang, G. et al. Vorinostat and sorafenib synergistically kill tumor cells via FLIP suppression and CD95 activation. Clin Cancer Res 14, 5385-5399 (2008); Castro, B. M., de Almeida, R. F., Goormaghtigh, E., Fedorov, A. & Prieto, M. Organization and dynamics of Fas transmembrane domain in raft membranes and modulation by ceramide. Biophys J 101, 1632-1641 (2011); Grassme, H. et al. CD95 signaling via ceramide-rich membrane rafts. J Biol Chem 276, 20589-20596 (2001); Gajate, C. & Mollinedo, F. Lipid raft-mediated Fas/CD95 apoptotic signaling in leukemic cells and normal leukocytes and therapeutic implications. J Leukoc Biol 98, 739-759, (2015); and Gajate, C. & Mollinedo, F. Lipid rafts and raft-mediated supramolecular entities in the regulation of CD95 death receptor apoptotic signaling. Apoptosis 20, 584-606 (2015)). Therefore, ceramide analogs have the potential to enhance Fas receptor aggregation and thus increase the efficacy of FasL-induced apoptosis.

Therefore it is an object of the invention to provide ceramide compositions for treating cancer, for example colorectal cancer.

It is another object of the invention to provide ceramide analogs effective to treat one or more symptoms of cancer.

It is still another embodiment of the invention to provide ceramide compositions and methods for sensitizing cancer or tumor cells to chemotherapy or radiation therapy.

### SUMMARY OF THE INVENTION

Ceramide analogs are provided that are useful for enhancing or promoting immune responses. The ceramide analogs described herein can be defined according to formula (I) as follows:

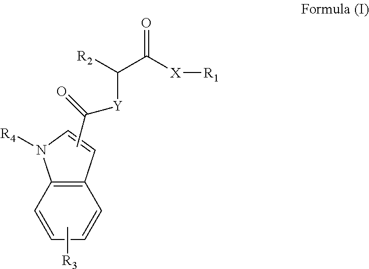

wherein X and Y are independently selected to be O, NH, S, or NR₅;

wherein R₁ is selected from C₁-C₃₀ linear, branched, or cyclic, optionally substituted alkyl, heteroalkyl, alkenyl, or alkynyl groups;

wherein R₂ is selected to be a hydrogen, C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₃ is selected from hydrogen, C₁-C₃₀ linear, branched, or cyclic, optionally substituted alkyl, aromatic, or heteroaromatic group or a halo substituent (i.e., F, Cl, Br, I), C₁-C₃₀ optionally substituted alkoxyl, or a nitro (—NO₂) group;

wherein R₄ is selected from hydrogen, C₁-C₃₀ linear or branched substituted or unsubstituted alkyl, alkenyl, or alkynyl groups; and

wherein R₅ is selected from C₁-C₃₀ linear or branched substituted or unsubstituted alkyl, alkenyl, or alkynyl groups.

In certain embodiments, substituent R₃ may independently substitute one or more positions of the benzene ring of the indole, as permitted by valency. In preferred embodiments of analogs according to formula (I), R₃ is selected to be a chloro, bromo, fluoro, methyl, nitro, or methoxy group. In other embodiments, R₃ can be a 3-,4-,5-membered aromatic or heteroaromatic ring attached to any position of the benzene ring of the indole, as permitted by valency. In some preferred embodiments, X and Y are NH. In other preferred embodiments, Y is NR₅, wherein R₅ is a methyl group. In some preferred embodiments R₄ is hydrogen. In yet other preferred embodiments, R₁ is selected to be a C₁-C₃₀ alkyl group, more preferably a C₅-C₂₀ alkyl, even more preferably a C₁₀-C₁₅ alkyl.

In some ceramide analog embodiments according to formula (I) the carbonyl groups shown in the formula may independently be replaced by bioisosteric groups. Such bioisosteric groups include, but are not limited to, C₁-C₁₀ linear, branched, or cyclic, optionally substituted alkyl; or C₁-C₁₀ optionally substituted heterocylic groups.

In some other embodiments, the analogs can be defined according to formula (II) as follows:

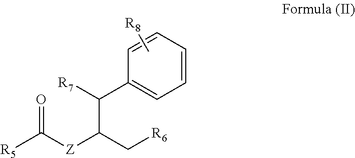

wherein Z selected to be O, NH, S, or NR₉;

wherein R₅ is selected from C₁-C₃₀ linear, branched, or cyclic, optionally substituted alkyl, heteroalkyl, alkenyl, or alkynyl groups;

wherein R₆ and R₇ are independently selected to be a hydrogen, hydroxyl, or C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₈ is selected from hydrogen, C₁-C₃₀ linear, branched, or cyclic, optionally substituted alkyl, halo substituent (i.e., F, Cl, Br, I), C₁-C₃₀ optionally substituted alkoxyl, or nitro (—NO₂) groups; and wherein R₉ is selected from C₁-C₃₀ linear or branched substituted or unsubstituted alkyl, alkenyl, or alkynyl groups.

In preferred embodiments of analogs according to formula (II), R₇ and R₈ are hydroxyl groups. In other embodiments, R₈ is selected to be a nitro, chloro, bromo, fluoro, methyl, or methoxy group. In certain embodiments, substituent R₈ may independently substitute one or more positions of the phenyl group, as permitted by valency. In some preferred embodiments, Z is NH. In yet other preferred embodiments, R₅ is selected to be a C₁-C₃₀ alkyl group, more preferably a C₅-C₂₀ alkyl, even more preferably a C₁₀-C₁₅ alkenyl group.

In certain embodiments, of the ceramide analog according to formula (II) the carbonyl group shown in the formula may be replaced by an bioisosteric group. Such bioisosteric groups include, but are not limited to, C₁-C₁₀ linear, branched, or cyclic, optionally substituted alkyl; or C₁-C₁₀ optionally substituted heterocylic groups.

In certain embodiments, the compounds are represented by formula (III) shown below:

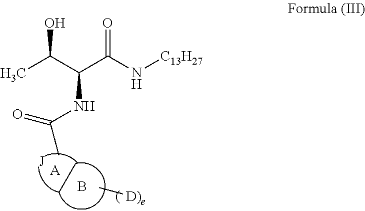

wherein

A is a C₃-C₇ ring containing a heteroatom J,

J includes, but is not limited to O, S, or NR9,

R₉ is hydrogen, or C₁-C₃₀, wherein C1-C30 is linear, branched substituted, or unsubstituted alkyl, alkenyl, or alkynyl groups,

R₁₀ is hydrogen, or C₁-C₃₀, wherein C1-C30 is linear, branched substituted, or unsubstituted alkyl, alkenyl, or alkynyl groups,

B is a ring system including, but not limited to C₃-C₇ cyclic aliphatic, aryl, or substituted aryl,

D is a chemical moiety including, but not limited to, a nitro group (—NO₂), halogen (F, Cl, Br, I), alkyl group, hydroxyl, etc. Preferably the alkyl group is a lower alkyl group containing C₁ to C₆ carbon atoms, such as methyl. e is an integer from 1-12.

In one embodiment, R₉ is preferably hydrogen or methyl.

In certain embodiments, J is a NH, and together with the A and B rings, forms an indole ring shown below:

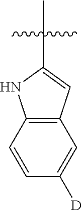

wherein D is —NO₂, Br, or CH₃.

The compounds of formulas (I), (II), and (III) described herein may possess asymmetric carbon atoms (chiral centers); the racemates, diastereomers, geometric isomers and individual isomers are within the scope of the compounds described herein. The compounds described herein may be prepared as a single isomer or as a mixture of isomers. In some embodiments, the compounds are prepared as a single isomer, substantially separated from other isomers. Methods of preparing substantially isomerically pure compounds are known in the art.

The compounds of formulas (I), (II), and (III) may be in their free acid or base form or may be pharmacologically acceptable salts thereof.

Representative ceramide analogs have the following structures:

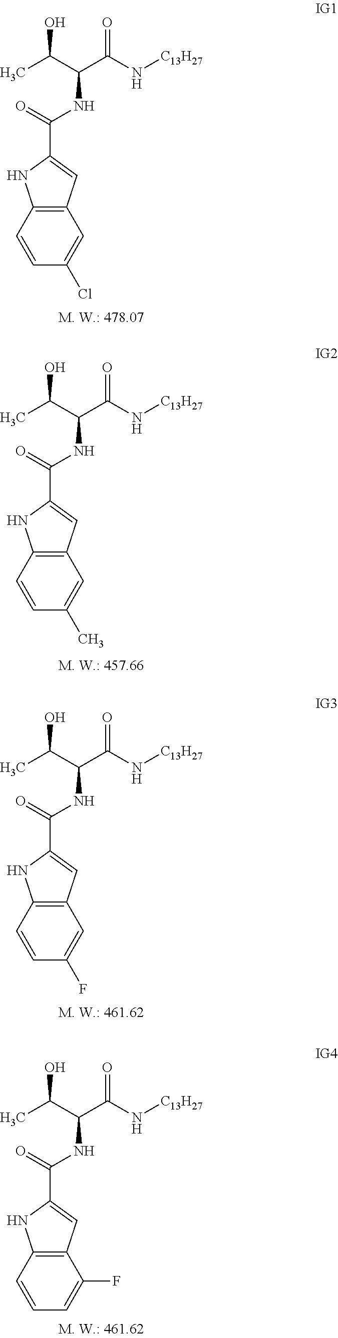

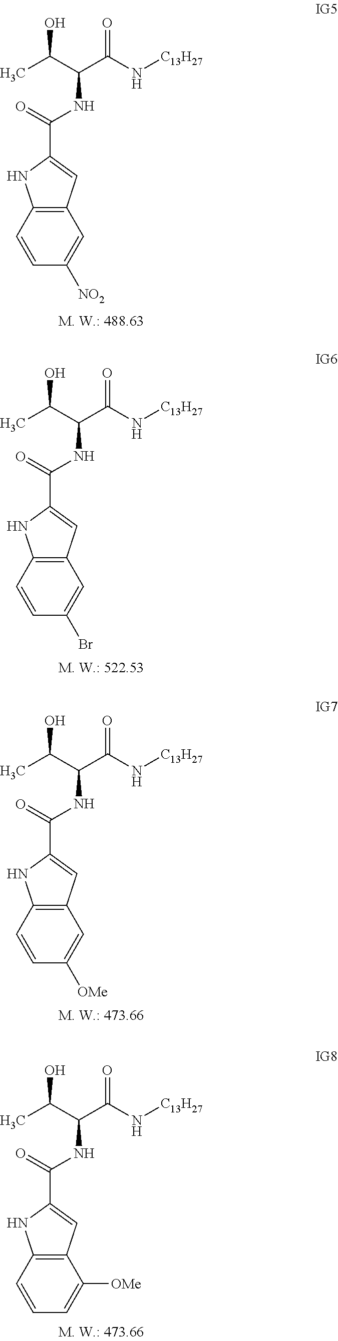

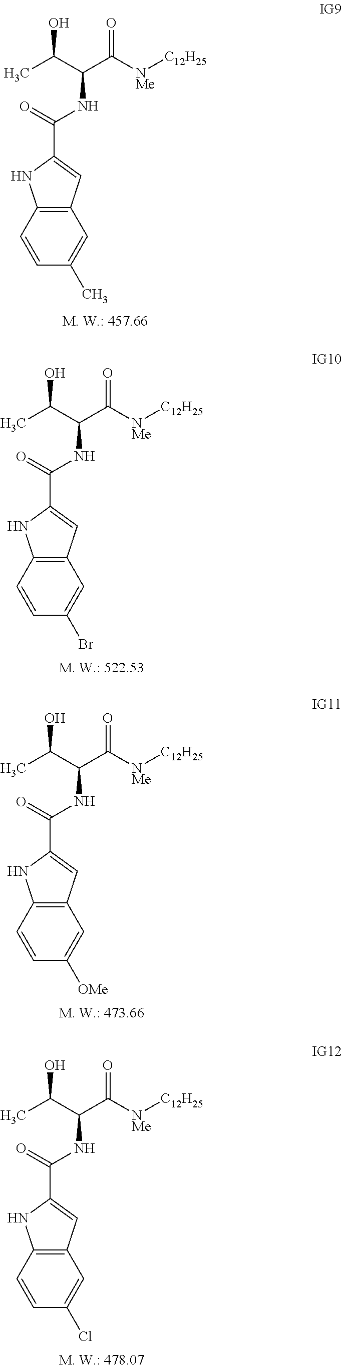

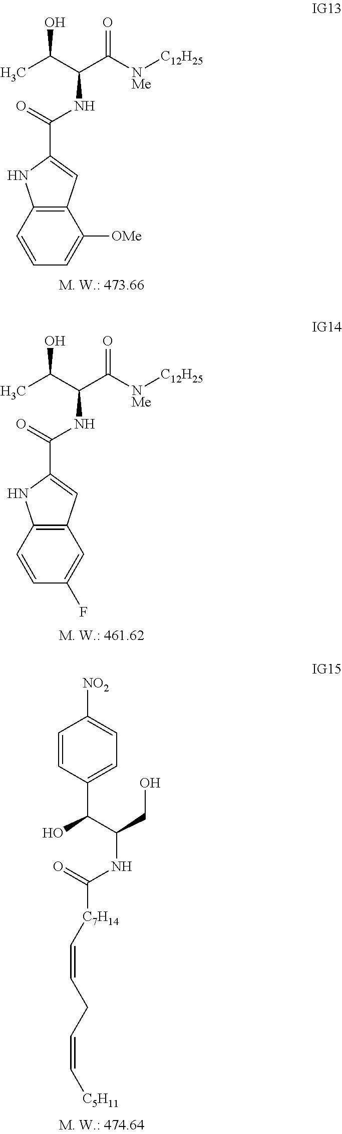

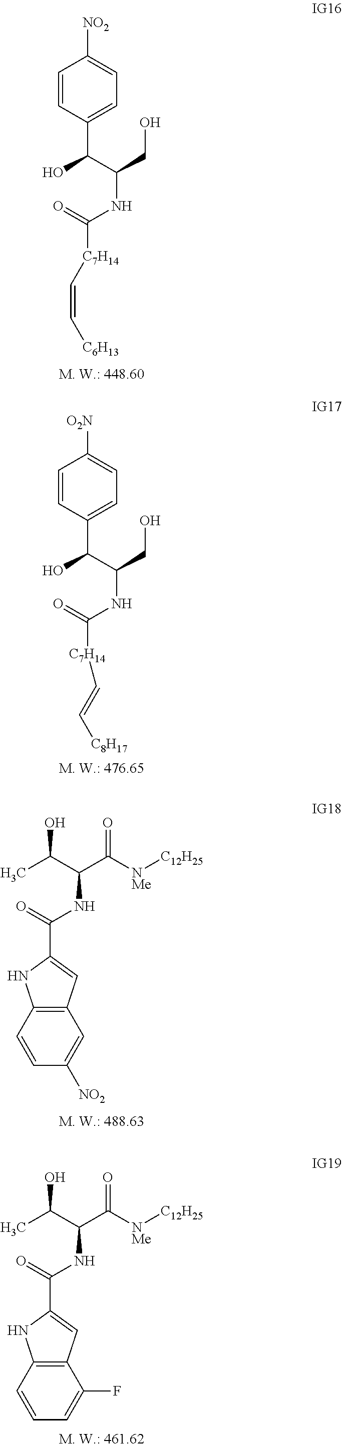

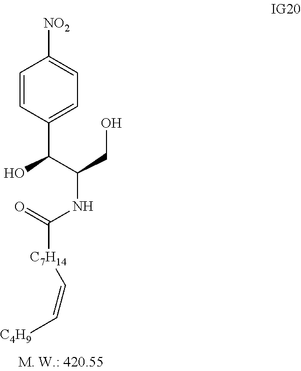

Additional ceramide analogs include N-((2S,3R)-3-hydroxy-1-(methyl(octadecyl)amino)-1-oxobutan-2-yl)-5H-[1,3]dioxolo[4,5-f]indole-6-carboxamide

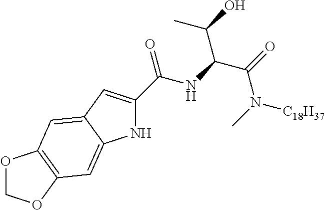

N-((2S,3R)-3-hydroxy-1-(methyl(octadecyl)amino)-1-oxobutan-2-yl)-5H-[1,3]dioxolo[4,5-f]indole-6-carboxamide
   Chemical Formula: C₃₃H₅₃N₃O₅
   Exact Mass: 571.40
   Molecular Weight: 571.80

### N-((2S,3R)-1-(dodecyl(methyl)amino)-3-hydroxy-1-oxobutan-2-yl)-5H-[1,3]dioxolo[4,5-f]indole-6-carboxamide

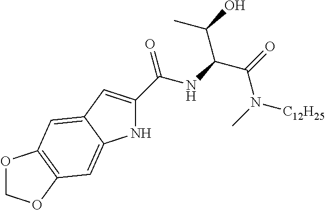 [0046] N-((2S,3R)-1-(dodecyl(methyl)amino)-3-hydroxy-1-oxobutan-2-yl)-5H-[1,3]dioxolo[4,5-f]indole-6-carboxamide
   Chemical Formula: C₂₇H₄₁N₃O₅
   Exact Mass: 487.30
   Molecular Weight: 487.64

### N-((2S,3R)-3-hydroxy-1-(methyl(tridecyl)amino)-1-oxobutan-2-yl)-5H-[1,3]dioxolo[4,5-f]indole-6-carboxamide

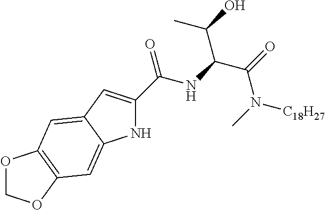 [0051] N-((2S,3R)-3-hydroxy-1-(methyl(tridecyl)amino)-1-oxobutan-2-yl)-5H-[1,3]dioxolo[4,5-f]indole-6-carboxamide
   Chemical Formula: C₂₈H₄₃N₃O₅
   Exact Mass: 501.32
   Molecular Weight: 501.67

### N-((1S,2R)-1,3-dihydroxy-1-(4-nitrophenyl)propan-2-yl)oleamide

One embodiment provides a pharmaceutical composition containing one or more of the ceramide analogs or a pharmaceutically acceptable salt, hydrate, enantiomer or stereoisomer thereof, together with a pharmaceutically acceptable excipient.

Still another embodiment provides a method for increasing cancer cell sensitivity to FasL-induced apoptosis by administering an effective amount of one or more ceramide analogs to cancer cells to enhance Fas oligomerization and to increase caspase 8 activity in the cancer cells. In one embodiment, the ceramide analog is selected from the group consisting of IG4, IG7, IG8, IG14, IG17 and IG19.

Another embodiment provides a method for increasing CTL-mediated and FasL-induced apoptosis of cancer cells in a subject by administering to a subject in need thereof an effective amount of a ceramide analog disclosed herein to increase cancer cell sensitivity to FasL-induced apoptosis. For example, a ceramide analog selected from the group consisting of IG4, IG7, IG8, IG14, IG17 and IG19.

Another embodiment provides a method for treating colon cancer by administering to a subject in need thereof an effective amount of a ceramide analog disclosed herein, for example selected from the group consisting of IG4, IG7, IG8, IG14, IG17 and IG19, to increase cancer cell sensitivity to FasL-induced apoptosis.

Yet another embodiment provides a method for improving the efficacy of a CTL-mediated immunotherapy by administering to a subject undergoing CTL-mediated immunotherapy an effective amount of a ceramide analog disclosed herein, for example, selected from the group consisting of IG4, IG7, IG8, IG14, IG17 and IG19 to increase cancer cell sensitivity to FasL-induced apoptosis.

### BRIEF DESCRIPTION OF THE DRAWINGS

FIGS. 1A-1T are microphotographs showing Fas protein level in normal human colon and human colorectal carcinoma tissues. Human colon carcinoma tissues were stained with anti-human Fas monoclonal antibody. Brown color indicates Fas protein level, with counterstaining by hematoxylin in blue. Shown are representative images of adjacent normal human colon tissues from colon cancer patients (each row of microphotographs are tissues from four patients), adenomas (FIGS. 1E-1H), primary invasive adenocarcinoma (FIGS. 1I-1L), colorectal adenocarcinoma metastatic to lymph nodes (FIGS. 1M-1P), and colorectal adenocarcinoma metastatic to liver (FIGS. 1Q-1T).

FIGS. 2A-2F are histograms showing Fas protein level on human colon carcinoma cell surface. In FIG. 1A SW480 human colon carcinoma cells were stained with IgG isotype control or anti-Fas mAbs and analyzed by flow cytometry. Shown are plots of each cell line. In FIG. 1B LS174T human colon carcinoma cells were stained with IgG isotype control or anti-Fas mAbs and analyzed by flow cytometry. FIG. 2C shows HCT116 human colon carcinoma cells were stained with IgG isotype control or anti-Fas mAbs and analyzed by flow cytometry. FIG. 2D shows HT29 human colon carcinoma cells were stained with IgG isotype control or anti-Fas mAbs and analyzed by flow cytometry, FIG. 2E shows RKO human colon carcinoma cells were stained with IgG isotype control or anti-Fas mAbs and analyzed by flow cytometry. FIG. 2F shows CACO2 human colon carcinoma cells were stained with IgG isotype control or anti-Fas mAbs and analyzed by flow cytometry. FIG. 2G is a bar graph the mean fluorescent intensity (MFI) of Fas of each cell line. Column: mean; Bar: SD.

FIGS. 3A-3F are line graphs showing sensitivity of human colon carcinoma cells to FasL-induced apoptosis. The indicated human colon carcinoma cells were cultured in the presence of MegaFasL at the indicated concentrations for approximately 24 h. Both floating and adherent cells were harvested and stained for Annexin V and PI. FIG. 3A is a line graph of Percent apoptotic cell death versus FasL (ng/ml) for SW480 cells. Percent apoptotic cell death was calculated as (% Annexin V⁺PI⁺ cells in the presence of FasL)−(% Annexin V⁺PI⁺ cells in the absence of FasL). FIG. 3B is a line graph of Percent apoptotic cell death versus FasL (ng/ml) for CACO2 cells. FIG. 3C is a line graph of Percent apoptotic cell death versus FasL (ng/ml) for HCT116 cells. FIG. 3D is a line graph of Percent apoptotic cell death versus FasL (ng/ml) for LS174T cells. FIG. 3E is a line graph of Percent apoptotic cell death versus FasL (ng/ml) for RKO cells. FIG. 3F is a line graph of Percent apoptotic cell death versus FasL (ng/ml) for HT29 cells. Column: mean; Bar: SD.

FIG. 4 is a bar graph of Percent Cell Viability (MTT assay) showing. cytotoxicity of the indicated ceramide analogs to human colon carcinoma cells. Human colon carcinoma SW480 cells were cultured in the presence of ceramide analogs at the indicated concentrations for three days. Cell viability was determined by MTT assays. % cell viability of control was set at 100% and cell viability of treatment groups was calculated as % of the control groups. IC₅₀ was calculated using GraphPad Prism program.

FIG. 5A is a bar graph of Percent Apoptotic cell death of SW480 cells treated with the indicated ceramide analogs (10 μM), with (10 ng/ml) or without FasL and treated with FasL alone. FIG. 5B is a bar graph of Percent Apoptotic cell death of RKO cells treated with the indicated ceramide analogs (10 μM), with (100 ng/ml) or without FasL and treated with FasL alone. FIG. 5C is a bar graph of Percent Apoptotic cell death of HCT cells treated with the indicated ceramide analogs (10 μM), with (10 ng/ml) or without FasL and treated with FasL alone. % apoptotic cell death was calculated as % Annexin V⁺PI⁺ cells in the presence of ceramide analogs plus MegaFasL−% Annexin V⁺PI⁺ cells in the control group. Column: mean; Bar: SD.

FIGS. 6A-6C are Western blots of human colon carcinoma SW480 (FIG. 6A), RKO (FIG. 6B) and HCT116 cells (FIG. 6C) cultured in the presence of the indicated ceramide analogs or ceramide analogs plus MegaFasL for 4 h. Cells were collected and lysed in cytosol buffer. Cytosolic fractions were resolved in 4-20% SDS polyacrylamide gel and analyzed by Western blotting using anti-active caspase 8 and anti-cleaved PARP antibodies, respectively. The membranes were stripped and re-probed with anti-β-actin antibody. The procaspase 8, cleaved caspase 8, cleaved PARP and β-actin are indicated at the right. The locations of molecular weight markers are indicated at the left.

FIG. 7A is a line graph of percent tumor cell lysis verus pfpCTLs E/T ratio. Cells were gated for CFSE⁺ tumor cells. The gated cells were then analyzed for PI⁺ cells. % tumor cell lysis was calculated as % CFSE⁺PI⁺ cells in the presence of CTLs−% CFSE⁺PI⁺ cells in the absence of CTLs. FIG. 7B is a bar graph of percent tumor cell lysis Cells were gated for CFSE⁺ tumor cells. The gated cells were then analyzed for PI⁺ cells. % tumor cell lysis was calculated as % CFSE⁺PI⁺ cells in the presence of ceramide analogs or ceramide analogs plus pfpCTLs−% CFSE⁺PI⁺ cells in the absence of ceramide analogs or ceramide analogs plus pfpCTLs. Column: mean; Bar: SD. Bolded ** indicated p<0.01 between ceramide analog+pfpCTLs group and pfpCTLs only group, and green ** indicates p<0.01 between ceramide analog+pfpCTLs group and ceramide analog only group. Column: mean; Bar: SD.

FIG. 8 is a bar graph of number of tumor nodules/lung in lungs taken from BALB/c mice injected with CT26 cells (2.5×10⁵ cell per mouse). Tumor-bearing mice were treated with the indicated 5 ceramide analogs (25 and 50 kg body weight) by intraperitoneal injection at days 8, 10 and 12 after tumor injection.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), preferably 20 or fewer, more preferably 10 or fewer, most preferably 6 or fewer. If the alkyl is unsaturated, the alkyl chain generally has from 2-30 carbons in the chain, preferably from 2-20 carbons in the chain, more preferably from 2-10 carbons in the chain. Likewise, preferred cycloalkyls have from 3-20 carbon atoms in their ring structure, preferably from 3-10 carbons atoms in their ring structure, most preferably 5, 6 or 7 carbons in the ring structure.

The terms “alkenyl” and “alkynyl” refer to unsaturated aliphatic groups analogous in length and possible substitution to the alkyls described above, but that contain at least one double or triple bond respectively.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

“Alkoxy”, “alkylamino”, and “alkylthio” are used herein in their conventional sense, and refer to those alkyl groups attached to the remainder of the molecule via an oxygen atom, an amino group, or a sulfur atom, respectively.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀)alkyl, phenyl or benzyl, and optionally containing 1-3 double bonds and optionally substituted with one or more substituents. One of the rings can be aromatic. Examples of heterocyclic and heteroaromatic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents as defined above for alkyl and aryl.

“Halogen” or “halo”, as used herein, refers to fluorine, chlorine, bromine, or iodine.

### II. Ceramide Analogs

The ceramide analogs described herein can be defined according to formula (I) as follows:

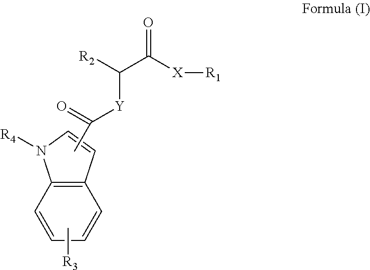

wherein X and Y are independently selected to be O, NH, S, or NR₅;

wherein R₁ is selected from C₁-C₃₀ linear, branched, or cyclic, optionally substituted alkyl, heteroalkyl, alkenyl, or alkynyl groups;

wherein R₂ is selected to be a hydrogen, C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₃ is selected from hydrogen, C₁-C₃₀ linear, branched, or cyclic, optionally substituted alkyl, aromatic, or heteroaromatic group or a halo substituent (i.e., F, Cl, Br, I), C₁-C₃₀ optionally substituted alkoxyl, or a nitro (—NO₂) group;

wherein R₄ is selected from hydrogen, C₁-C₃₀ linear or branched substituted or unsubstituted alkyl, alkenyl, or alkynyl groups; and

wherein R₅ is selected from C₁-C₃₀ linear or branched substituted or unsubstituted alkyl, alkenyl, or alkynyl groups.

In certain embodiments, substituent R₃ may independently substitute one or more positions of the benzene ring of the indole, as permitted by valency. In preferred embodiments of analogs according to formula (I), R₃ is selected to be a chloro, bromo, fluoro, methyl, nitro, or methoxy group. In other embodiments, R₃ can be a 3-,4-,5-membered aromatic or heteroaromatic ring attached to any position of the benzene ring of the indole, as permitted by valency. In some preferred embodiments, X and Y are NH. In other preferred embodiments, Y is NR₅, wherein R₅ is a methyl group. In some preferred embodiments R₄ is hydrogen. In yet other preferred embodiments, R₁ is selected to be a C₁-C₃₀ alkyl group, more preferably a C₅-C₂₀ alkyl, even more preferably a C₁₀-C₁₅ alkyl.

In some ceramide analog embodiments according to formula (I) the carbonyl groups shown in the formula may independently be replaced by bioisosteric groups. Such bioisosteric groups include, but are not limited to, C₁-C₁₀ linear, branched, or cyclic, optionally substituted alkyl; or C₁-C₁₀ optionally substituted heterocylic groups.

In some other embodiments, the analogs can be defined according to formula (II) as follows:

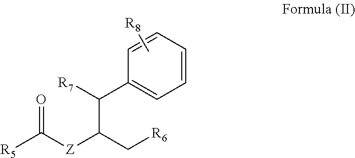

wherein Z selected to be O, NH, S, or NR₉;

wherein R₅ is selected from C₁-C₃₀ linear, branched, or cyclic, optionally substituted alkyl, heteroalkyl, alkenyl, or alkynyl groups;

wherein R₆ and R₇ are independently selected to be a hydrogen, hydroxyl, or C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₈ is selected from hydrogen, C₁-C₃₀ linear, branched, or cyclic, optionally substituted alkyl, halo substituent (i.e., F, Cl, Br, I), C₁-C₃₀ optionally substituted alkoxyl, or nitro (—NO₂) groups; and wherein R₉ is selected from C₁-C₃₀ linear or branched substituted or unsubstituted alkyl, alkenyl, or alkynyl groups.

In preferred embodiments of analogs according to formula (II), R₇ and R₈ are hydroxyl groups. In other embodiments, R₈ is selected to be a nitro, chloro, bromo, fluoro, methyl, or methoxy group. In certain embodiments, substituent R₈ may independently substitute one or more positions of the phenyl group, as permitted by valency. In some preferred embodiments, Z is NH. In yet other preferred embodiments, R₅ is selected to be a C₁-C₃₀ alkyl group, more preferably a C₅-C₂₀ alkyl, even more preferably a C₁₀-C₁₅ alkenyl group.

In certain embodiments, of the ceramide analog according to formula (II) the carbonyl group shown in the formula may be replaced by an bioisosteric group. Such bioisosteric groups include, but are not limited to, C₁-C₁₀ linear, branched, or cyclic, optionally substituted alkyl; or C₁-C₁₀ optionally substituted heterocylic groups.

In certain embodiments, the compounds are represented by formula (III) shown below:

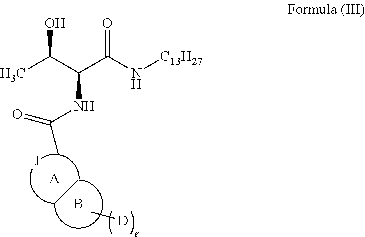

wherein

A is a C₃-C₇ ring containing a heteroatom J,

J includes, but is not limited to O, S, or NR9,

R₉ is hydrogen, or C₁-C₃₀, wherein C1-C30 is linear, branched substituted, or unsubstituted alkyl, alkenyl, or alkynyl groups,

R₁₀ is hydrogen, or C₁-C₃₀, wherein C1-C30 is linear, branched substituted, or unsubstituted alkyl, alkenyl, or alkynyl groups,

B is a ring system including, but not limited to C₃-C₇ cyclic aliphatic, aryl, or substituted aryl,

D is a chemical moiety including, but not limited to, a nitro group (—NO₂), halogen (F, Cl, Br, I), alkyl group, hydroxyl, etc. Preferably the alkyl group is a lower alkyl group containing C₁ to C₆ carbon atoms, such as methyl. e is an integer from 1-12.

In one embodiment, R₉ is preferably hydrogen or methyl.

In certain embodiments, J is a NH, and together with the A and B rings, forms an indole ring shown below:

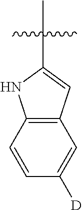

wherein D is —NO₂, Br, or CH₃.

The ceramide analog compounds of formulas (I), (II), and (III) described herein may possess asymmetric carbon atoms (chiral centers); the racemates, diastereomers, geometric isomers and individual isomers are within the scope of the compounds described herein. The compounds described herein may be prepared as a single isomer or as a mixture of isomers. In some embodiments, the compounds are prepared as a single isomer, substantially separated from other isomers. Methods of preparing substantially isomerically pure compounds are known in the art.

The ceramide analog compounds of formulas (I), (II), and (III) may be in their free acid or base form or may be pharmacologically acceptable salts thereof.

Twenty-three exemplary ceramide analogs according to formulas (I), (II), and (III) were developed and six ceramide analogs were functionally characterized and shown to effectively increase carcinoma cell sensitivity to FasL-induced apoptosis at sublethal doses. The structures of the analogs are provided in Table S1 and in the Examples.

TABLE S1
Ceramide analogs and their molecular weights.

| | |
|---|---|
| 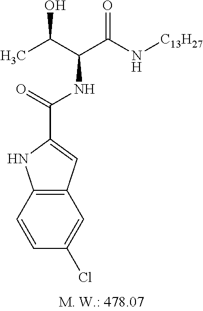 | IG1 |
| 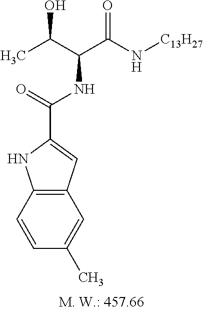 | IG2 |
| 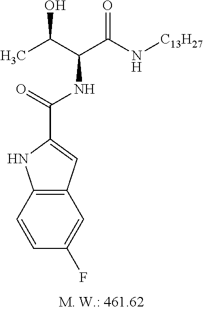 | IG3 |
| 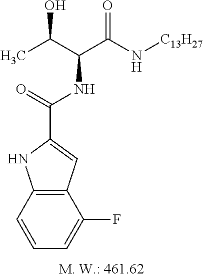 | IG4 |
| 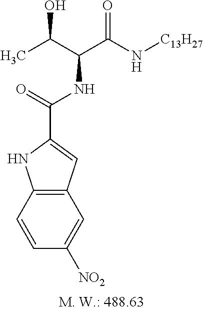 | IG5 |
| 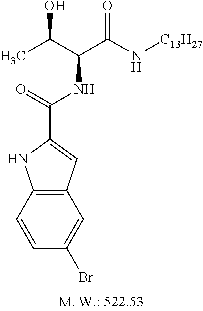 | IG6 |
| 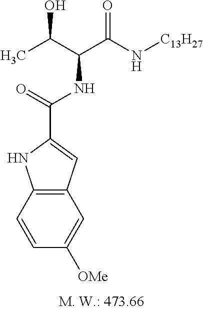 | IG7 |
| 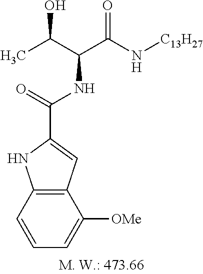 | IG8 |
| 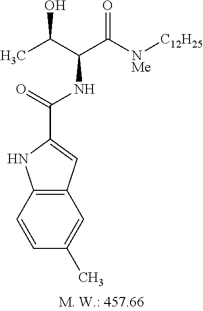 | IG9 |
| 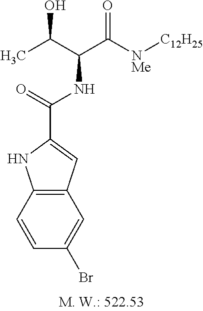 | IG10 |
| 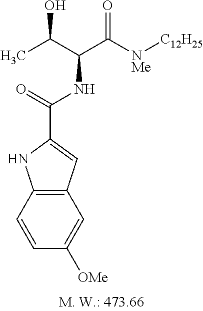 | IG11 |
| 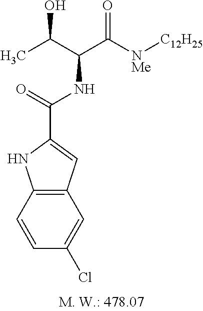 | IG12 |
| 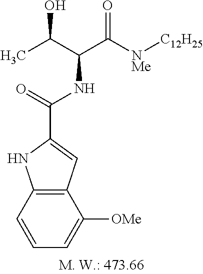 | IG13 |
| 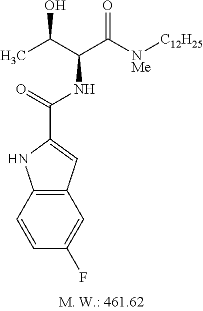 | IG14 |
| 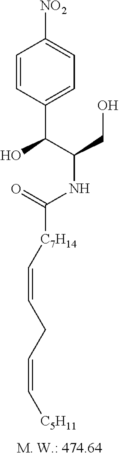 | IG15 |
| 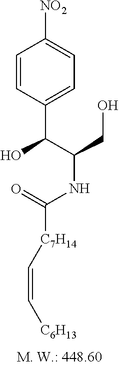 | IG16 |
| 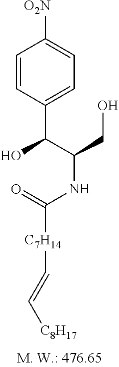 | IG17 |
| 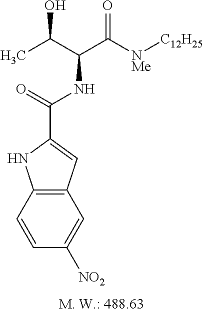 | IG18 |
| 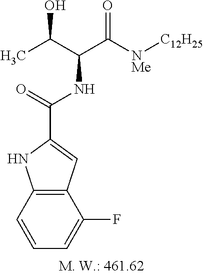 | IG19 |
| 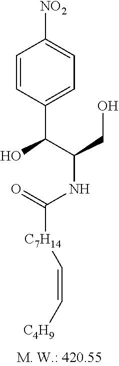 | IG20 |

These six ceramide analogs that can effectively increase human colon carcinoma cell sensitivity to FasL-induced apoptosis at sublethal doses are IG4, IG7, IG8, IG14, IG17 and IG19. More importantly, these ceramide analogs also effectively increased colon carcinoma cell sensitivity to FasL-mediated cytotoxicity by tumor-specific CTLs.

CTL-based cancer immunotherapies, including CTL adoptive transfer, check point blockade (anti-PD-1 and anti-CTLA4 mAb) and CART cell immunotherapy have recently shown remarkable and durable efficacy in suppression of various human cancers in the clinics Dudley, M. E. et al. Adoptive cell transfer therapy following non-myeloablative but lymphodepleting chemotherapy for the treatment of patients with refractory metastatic melanoma. J Clin Oncol 23, 2346-2357 (2005); Garfall, A. L., Stadtmauer, E. A. & June, C. H. Chimeric Antigen Receptor T Cells in Myeloma. N Engl J Med 374, 194, doi:10.1056/NEJMc1512760 (2016); and Brahmer, J. R. et al. Safety and activity of anti-PD-L1 antibody in patients with advanced cancer. N Engl J Med 366, 2455-2465, doi:10.1056/NEJMoa1200694 (2012). However, the patient objective response rate for anti-PD-1 immunotherapy is only about 6-17% Brahmer, J. R. et al. Safety and activity of anti-PD-L1 antibody in patients with advanced cancer. N Engl J Med 366, 2455-2465, doi:10.1056/NEJMoa1200694 (2012). All of these immunotherapies depend on CTL-induced target tumor cell apoptosis. Apoptosis resistance of cancer cells, either intrinsic or acquired, is a hallmark of human cancer Hanahan, D. & Weinberg, R. A. Hallmarks of cancer: the next generation. Cell 144, 646-674, doi:S0092-8674(11)00127-9 [pii]10.1016/j.cell.2011.02.013 (2011). Therefore, if cancer cells are not sensitive to apoptosis, regardless how potent the CTLs are, the target tumor cell lysis efficacy of immunotherapy is not going to be high. It is known that CTLs kill target cells primarily through two effector mechanisms: the perforin-mediated and Fas-mediated cytotoxicity Kagi, D. et al. Fas and perforin pathways as major mechanisms of T cell-mediated cytotoxicity. Science 265, 528-530 (1994). The disclosed ceramide analogs can significantly increase colon carcinoma cell sensitivity to FasL-induced apoptosis of tumor-specific CTLs. These ceramide analogs may thus have the potential to be translated as adjunct agents to increase the efficacy of CTL adoptive transfer, check point blockade and CAR T cell immunotherapy.

In addition to cancer cell apoptosis resistance, immune suppression is another major impediment in CTL-based cancer immunotherapy Ostrand-Rosenberg, S. Myeloid-derived suppressor cells: more mechanisms for inhibiting antitumor immunity. Cancer Immunol Immunother 59, 1593-1600, doi:10.1007/s00262-010-0855-8 (2010). Although antigen-specific CTLs use both perforin-mediated and FasL-mediated cytotoxicity to kill target tumor cells under physiological conditions Caldwell, S. A., Ryan, M. H., McDuffie, E. & Abrams, S. I. The Fas/Fas ligand pathway is important for optimal tumor regression in a mouse model of CTL adoptive immunotherapy of experimental CMS4 lung metastases. J Immunol 171, 2402-2412 (2003); and Kagi, D. et al. Fas and perforin pathways as major mechanisms of T cell-mediated cytotoxicity. Science 265, 528-530 (1994). Recent studies showed that the immune suppressive Treg cells selectively inhibits the perforin-mediated cytotoxicity without affecting T cell activation Chen, M. L. et al. Regulatory T cells suppress tumor-specific CD8 T cell cytotoxicity through TGF-beta signals in vivo. Proc Natl Acad Sci USA 102, 419-424 (2005); and Mempel, T. R. et al. Regulatory T cells reversibly suppress cytotoxic T cell function independent of effector differentiation. Immunity 25, 129-141 (2006). Therefore, the FasL-mediated cytotoxicity of tumor-specific CTLs should still be active in the immune suppressive tumor microenvironment. The newly developed ceramide analogs effectively increase the efficacy of FasL-mediated target colon cancer cell lysis by tumor-specific CTLs suggest that these ceramide analogs may have the potential to increase CTL efficacy against immune suppressive cancers.

It is well-documented that ceramide also mediates the expression of apoptosis-regulatory genes and apoptosis pathways Bielawska, A. et al. Novel analogs of D-e-MAPP and B13. Part 2: signature effects on bioactive sphingolipids. Bioorg Med Chem 16, 1032-1045 (2008); Paschall, A. V et al. Ceramide targets xIAP and cIAP1 to sensitize metastatic colon and breast cancer cells to apoptosis induction to suppress tumor progression. BMC Cancer 14, 24, doi:10.1186/1471-2407-14-241471-2407-14-24 [pii] (2014); Beverly, L. J. et al. BAK activation is necessary and sufficient to drive ceramide synthase-dependent ceramide accumulation following inhibition of BCL2-like proteins. Biochem J 452, 111-119, doi:10.1042/BJ20130147BJ20130147 [pii] (2013); Debret, R. et al. Ceramide inhibition of MMP-2 expression and human cancer bronchial cell invasiveness involve decreased histone acetylation. Biochim Biophys Acta 1783, 1718-1727 (2008); Senkal, C. E., Ponnusamy, S., Bielawski, J., Hannun, Y. A. & Ogretmen, B. Antiapoptotic roles of ceramide-synthase-6-generated C16-ceramide via selective regulation of the ATF6/CHOP arm of ER-stress-response pathways. Faseb J 24, 296-308 (2010); and Chalfant, C. E. et al. De novo ceramide regulates the alternative splicing of caspase 9 and Bcl-x in A549 lung adenocarcinoma cells. Dependence on protein phosphatase-1. J Biol Chem 277, 12587-12595 (2002); Senkal, C. E. et al. Alteration of ceramide synthase 6/C16-ceramide induces activating transcription factor 6-mediated endoplasmic reticulum (ER) stress and apoptosis via perturbation of cellular Ca2+ and ER/Golgi membrane network. J Biol Chem 286, 42446-42458, doi:M111.287383 [pii] 10.1074/jbc.M111.287383 (2011); von Haefen, C. et al. Ceramide induces mitochondrial activation and apoptosis via a Bax-dependent pathway in human carcinoma cells. Oncogene 21, 4009-4019, doi:10.1038/sj.onc.1205497 (2002); Sauane, M. et al. Ceramide plays a prominent role in MDA-7/IL-24-induced cancer-specific apoptosis. J Cell Physiol 222, 546-555, doi:10.1002/jcp.21969 (2010); and Casson, L. et al. Inhibition of ceramide metabolism sensitizes human leukemia cells to inhibition of BCL2-like proteins. PLoS One 8, e54525, doi:10.1371/journal.pone.0054525. It is possible that the ceramide analogs may mediate the expression of these apoptosis regulatory genes and apoptosis pathways in human colon carcinoma cells. Nevertheless, the observation that these ceramide analogs enhance FasL-induced caspase 8 activation suggests that these ceramide analogs effectively mediate the Fas receptor DISC complex conformation to increase colon cancer cell sensitivity to apoptosis induction by T cells, which provides the molecular mechanism and strong rationale for further development of these ceramide analogs as adjunct agents in cancer immunotherapy.

Additional ceramide analogs include N-((2S,3R)-3-hydroxy-1-(methyl(octadecyl)amino)-1-oxobutan-2-yl)-5H-[1,3]dioxolo[4,5-f]indole-6-carboxamide

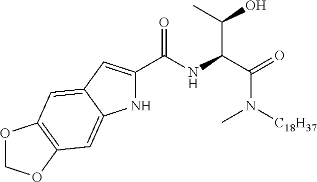

N-((2S,3R)-3-hydroxy-1-(methyl(octadecyl)amino)-1-oxobutan-2-yl)-5H-[1,3]dioxolo[4,5-f]indole-6-carboxamide
   Chemical Formula: C₃₃H₅₃N₃O₅
   Exact Mass: 571.40
   Molecular Weight: 571.80

### N-((2S,3R)-1-(dodecyl(methyl)amino)-3-hydroxy-1-oxobutan-2-yl)-5H-[1,3]dioxolo[4,5-f]indole-6-carboxamide

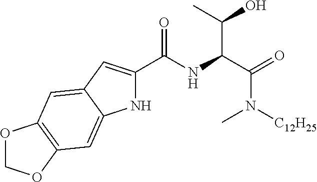 [0116] N-((2S,3R)-1-(dodecyl(methyl)amino)-3-hydroxy-1-oxobutan-2-yl)-5H-[1,3]dioxolo[4,5-f]indole-6-carboxamide
   Chemical Formula: C₂₇H₄₁N₃O₅
   Exact Mass: 487.30
   Molecular Weight: 487.64

### N-((2S,3R)-3-hydroxy-1-(methyl(tridecyl)amino)-1-oxobutan-2-yl)-5H-[1,3]dioxolo[4,5-f]indole-6-carboxamide

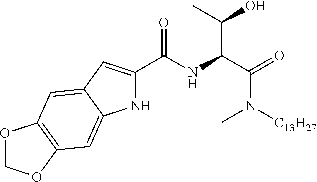 [0121] N-((2S,3R)-3-hydroxy-1-(methyl(tridecyl)amino)-1-oxobutan-2-yl)-5H-[1,3]dioxolo[4,5-f]indole-6-carboxamide
Chemical Formula: C₂₅H₄₃N₃O₅
Exact Mass: 501.32
Molecular Weight: 501.67

### N-((1S,2R)-1,3-dihydroxy-1-(4-nitrophenyl)propan-2-yl)oleamide

### III. Formulations

Formulations of and pharmaceutical compositions including one or more of the disclosed compounds are provided. Dosage ranges for specific small molecules are discussed above based on pre-clinical and clinical trial data. Generally, dosage levels, for the compounds disclosed herein are between about 0.0001 mg/kg of body weight to about 1,000 mg/kg, more preferably of 0.001 to 500 mg/kg, more preferably 0.01 to 50 mg/kg of body weight daily are administered to mammals. In some embodiments, polypeptides or nucleic acids are administered in a dosage of 0.01 to 50 mg/kg of body weight daily, preferably about 0.1 to 20 mg/kg. In some embodiments, nucleic acid dosages can range from about 0.001 mg to about 1,000 mg, more preferable about 0.01 mg to about 100 mg per administration (e.g., daily; or once, twice, or three times weekly, etc.).

1. Delivery Vehicles

The compounds can be administered and taken up into the cells of a subject with or without the aid of a delivery vehicle. Appropriate delivery vehicles for the disclosed active agents are known in the art and can be selected to suit the particular active agent. For example, in some embodiments, the compound is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the compound out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, the compounds are incorporated into the same particles and are formulated for release at different times and/or over different time periods. For example, in some embodiments, one of the compounds is released entirely from the particles before release of the second compound begins. In other embodiments, release of the first compound begins followed by release of the second compound before the all of the first compound is released. In still other embodiments, both compounds are released at the same time over the same period of time or over different periods of time.

The compounds can be incorporated into a delivery vehicle prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C. The release point and/or period of release can be varied as discussed above.

2. Pharmaceutical Compositions

Pharmaceutical compositions including the disclosed compounds, with or without a delivery vehicle, are provided. Pharmaceutical compositions can be for administration by parenteral (intramuscular, intraperitoneal, intravenous (IV) or subcutaneous injection), enteral, transmucosal (nasal, vaginal, rectal, or sublingual), or transdermal (either passively or using iontophoresis or electroporation) routes of administration or using bioerodible inserts and can be formulated in dosage forms appropriate for each route of administration.

In certain embodiments, the compositions are administered locally, for example by injection directly into a site to be treated (e.g., into a tumor). In some embodiments, the compositions are injected or otherwise administered directly into the vasculature onto vascular tissue at or adjacent to the intended site of treatment (e.g., adjacent to a tumor). Typically, local administration causes an increased localized concentration of the compositions which is greater than that which can be achieved by systemic administration.

a. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

b. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers. In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit®RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

c. Formulations for Pulmonary and Mucosal Administration

Active agent(s) and compositions thereof can be applied formulated for pulmonary or mucosal administration. The administration can include delivery of the composition to the lungs, nasal, oral (sublingual, buccal), vaginal, or rectal mucosa.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

d. Transdermal

Transdermal formulations may also be prepared. These will typically be ointments, lotions, sprays, or patches, all of which can be prepared using standard technology. Transdermal formulations can include penetration enhancers.

### IV. Methods of Using Ceramide Analogs

The disclosed ceramide analogs can be used to promote or enhance immune responses in subjects in need of such treatment. One embodiment provides a method for increasing cancer cell sensitivity to FasL-induced apoptosis by administering an effective amount of one or more ceramide analogs described herein to cancer cells to enhance Fas oligomerization and to increase caspase 8 activity in the cancer cells. Typically, the ceramide analog is administered to a patient or subject that has cancer. Cancers that can be treated with the disclosed ceramide analogs include but are not limited to cancers that form tumors. Exemplary cancers that can be treated include, but are not limited to head and neck cancer, brain cancer, bone cancer, skin cancer, liver cancer, soft tissue sarcoma, cancer of the reproductive organs, lung cancer, mouth cancer, colon cancer, and cancers of the digestive system. Preferred ceramide analogs used to treat cancer include, but are not limited to IG4, IG7, IG8, IG14, IG17 and IG19.

Another embodiment provides a method for increasing CTL-mediated and FasL-induced apoptosis of cancer cells in a subject by administering to a subject in need thereof an effective amount of a ceramide analog selected from the group consisting of IG4, IG7, IG8, IG14, IG17 and IG19 to increase cancer cell sensitivity to FasL-induced apoptosis.

In still other embodiments, the disclosed ceramide analogs are used in combination or alternation with other therapies including chemotherapy, radiation therapy, and CTL-based cancer immunotherapies, including CTL adoptive transfer, check point blockade (anti-PD-1 and anti-CTLA4 mAb) and CAR T cell immunotherapy. Generally, a subject receiving CTL-based immunotherapies is also administered an effective amount of ceramide analog to increase CTL-mediated and FasL-induced apoptosis. Thus, one or more ceramide analogs may be administered to a subject as an adjuvant therapy for a CTL-immunotherapy.

### EXAMPLES

### Materials and Methods

### Human Colon Cancer Cells.

Human colon cancer cell lines SW480, LS174T, HCT116, HT29, RKO, and CACO2 were obtained from American Type Culture Collection (ATCC) (Manassas, Va.). ATCC has characterized these cells by morphology, immunology, DNA fingerprint, and cytogenetics. All cells are cultured in RPMI medium plus 10% fetal bovine serum.

### Cell Viability Assays.

Cells were seeded in 96-well plates at 2×10³ cells/well in 100 μl culture medium for 3 days. Cell viability assays were performed using the MTT cell proliferation assay kit (ATCC, Manassas, Va.) according to the manufacturer's instructions.

### Flow Cytometry.

Cells were stained with fluorescent dye-conjugated anti-human Fas (Clone: DX2, Biolegend, San Diego, Calif.). Cells were then analyzed by flow cytometry.

### Immunohistochemistry.

Human Colon cancer tissue microarray slides were provided by the Cooperative Human Tissue Network (Mid-Atlantic Division, University of Virginia, Charlottesville, Va.). The tissues were stained with anti-human Fas (Clone: B-10, Santa Cruz Biotech, Dallas, Tex.). Slides were counterstained with hematoxylin (Richard-Allan Scientific, Kalamazoo, Mich.). Immunohistochemical staining was performed at the Georgia Pathology Services.

### Tumor Cell Apoptosis Assay.

Tumor cells were cultured in the presence of MegaFasL at the indicated concentrations as previously described Hu, X. et al. Deregulation of apoptotic factors Bcl-xL and Bax confers apoptotic resistance to myeloid-derived suppressor cells and contributes to their persistence in cancer. J Biol Chem 288, 19103-19115, doi:10.1074/jbc.M112.434530M112.434530 [pii] (2013). FasL (Mega-Fas Ligand®, kindly provided by Drs. Steven Butcher and Lars Damstrup at Topotarget A/S, Denmark) is a recombinant fusion protein that consists of three human FasL extracellular domains linked to a protein backbone comprising the dimmer-forming collagen domain of human adiponectin. The Mega-Fas Ligand was produced as a glycoprotein in mammalian cells using Good Manufacturing Practice compliant process in Topotarget A/S (Copenhagen, Denmark). For tumor cell apoptosis analysis, cells were stained with Alexa Fluor 647 Annexin V (Biolegend) in Annjixin V-binding buffer (10 mM Hepes, pH 7.4, 140 mM NaCl, 2.5 mM CaCl2) for 30 min at 4° C. Propidium Iodide was then added to the cell suspension, and cells were analyzed by flow cytometry as previously described Bardhan, K. et al. IFNgamma induces DNA methylation-silenced GPR109A expression via pSTAT1/p300 and H3K18 acetylation in colon cancer. Cancer Immunol Res, doi:canimm.0164.2014 [pii]2326-6066.CIR-14-0164 [pii] 10.1158/2326-6066.CIR-14-0164 (2015).

### Western Blotting Analysis.

Western blotting analysis was performed as previously described Paschall, A. V. et al. H3K9 Trimethylation Silences Fas Expression To Confer Colon Carcinoma Immune Escape and 5-Fluorouracil Chemoresistance. J Immunol, doi:1402243 [pii]jimmunol.1402243 [pii] 10.4049/jimmunol.1402243 (2015). Briefly, tumor cells were cultured in the presence of the indicated ceramide analogs or ceramide analogs plus MegaFasL for 4 h. Cells were collected and lysed in cytosol buffer [10 mM Hepes, pH 7.4, 250 mM Sucrose, 70 mM KCl, 1.5 mM MgCl2, 1 mM EDTA, 1 mM EGTA, protease and phosphatase inhibitor cocktails (Calbiochem, Billerica, Mass.), and 0.01% digitonin] for 10 min. Cytosolic fractions were resolved in 4-20% SDS polyacrylamide gel and analyzed by Western blotting. Anti-cleaved caspase 8 was obtained from R&D systems (AF705, R&D Systems. Minneapolis, Minn.). Anti-cytochromic C was obtained from BD Biosciences (Clone: 2H8, 2C12. San Diego, Calif.). Anti-cleaved human PARP antibody was obtained from Cell Signaling (Clone: D214, Danvers, Mass.). β-actin was obtained from Sigma-Aldrich (Clone: AC-15, St Louis, Mo.).

### CTL Cytotoxicity Assays.

Perforin-deficient CTLs were generated and maintained by weekly stimulation with AH1 peptide as previously described Liu, K., Caldwell, S. A., Greeneltch, K. M., Yang, D. & Abrams, S. I. CTL Adoptive Immunotherapy Concurrently Mediates Tumor Regression and Tumor Escape. J Immunol 176, 3374-3382 (2006). CT26 cells were labeled with CellTrace CFSE cell proliferation dye (C34554, Molecular Probes, Eugene, Oreg.) according to the manufacturer's instructions. Briefly, CFSE stock solution (in DMSO, Fisher-Thermal Scientific) was diluted with PBS to a working solution of 0.2 μM. Cells were resuspended in pre-warmed CFSE working solution and incubated at 37° C. for 15 min in the incubator. Cells were pelleted by centrifugation, resuspended in pre-warmed (37° C.) RPMI medium and incubated in the incubator for 30 min. Cells were then pelleted and resuspended in medium to a density of 4×10⁵ cells/ml. Transfer 50 ml labeled CT26 cells to each well of a U-bottom 96-well plate. CTLs were purified with the Lymphocytes Separation Medium, washed in medium and added to the tumor cell cultures at various ratios. The tumor-CTL mixtures were cultured in the CO2 incubator for approximately 24 h. Culture supernatant was collected. Adherent tumor cells were harvested using 0.05% Trypsin-EDTA solution and combined with the cultured supernatant. The collected tumor cell and CTL mixtures was pelleted by centrifugation, resuspended in PBS and stained with PI. Cells were analyzed immediately by flow cytometry. CFSE⁺ tumor cells were gated and analyzed for PI⁺ cells.

### Statistical Analysis.

Student's t test was also used to compare differences between different treatment groups. A p<0.05 was taken as statistically significant.

### Example 1: Fas Protein Level Decreases as Cancer Progresses in Human Colon Carcinoma

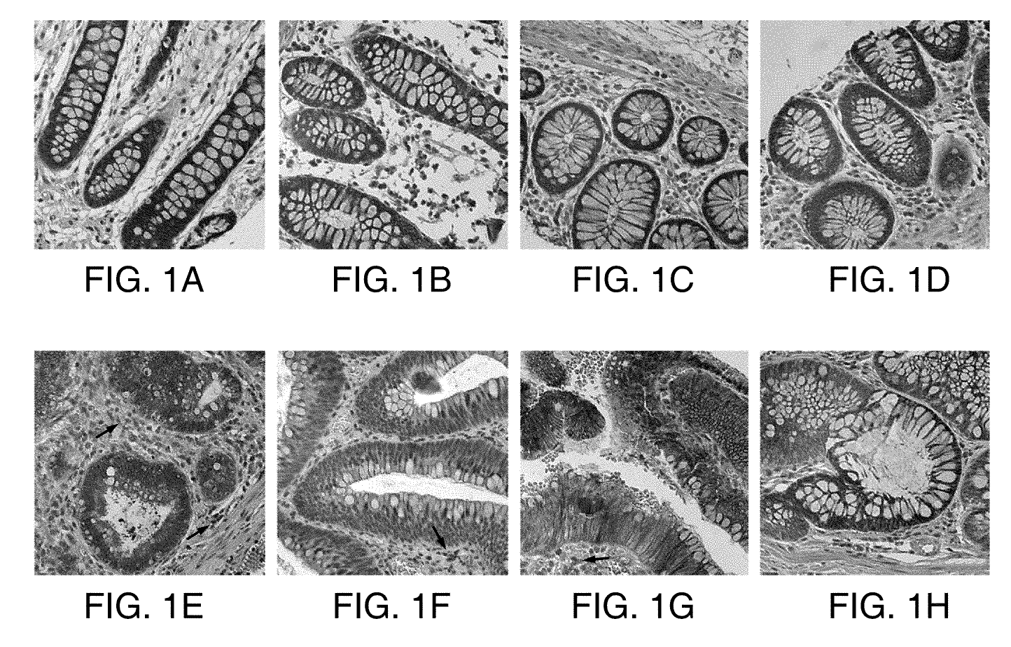

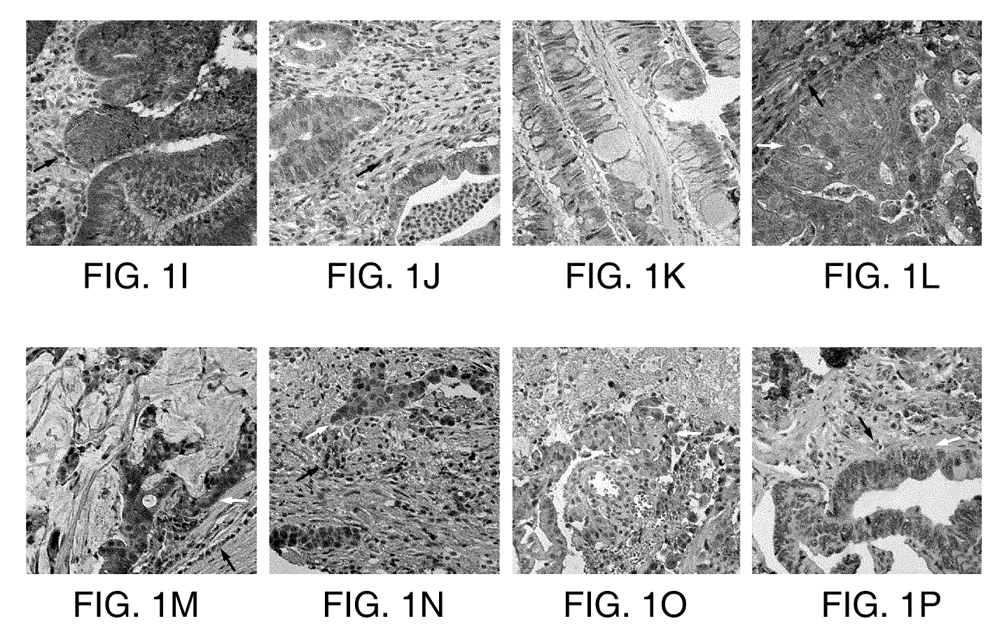

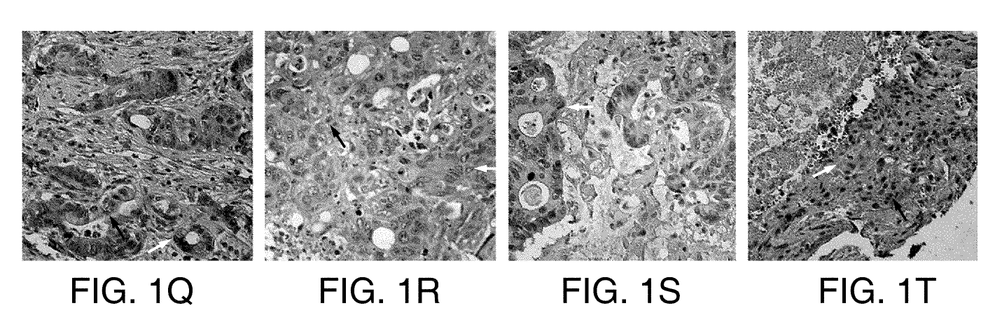

To determine Fas protein levels in normal colonic epithelial and colon carcinoma cells, adjacent normal colon tissues from human colon cancer patients were stained with human Fas-specific antibody by immunohistochemical methods. Fas protein level is high in all five normal colon tissues from five colon cancer patients (FIG. 1 and Table 1). Fas protein level in nine of the fourteen adenomas analyzed is as high as in normal colon tissues. The remaining five specimens showed medium levels of Fas protein (FIG. 1E-1H and Table 1). For the fourteen adenocarcinoma specimens analyzed, Fas protein levels range from high to low. Approximately 36% of specimens are high in Fas, 29% have medium Fas protein level, whereas about 36% exhibit low to undetectable Fas protein levels (FIG. 1I-1L and Table 1). Among the five lymph node (LN) metastases specimens analyzed, Fas protein levels showed similar patterns as the adenocarcinomas (FIG. 1M-1P and Table 1). Fas protein level is lower overall in the liver metastases specimens, with six of the seven specimens exhibiting low to undetectable Fas protein and only one liver metastases showing medium level of Fas protein (FIG. 1Q-1T and Table 1). Overall, the data indicates that Fas protein level decreases as colon cancer progresses.

TABLE 1
Fas protein level in normal human colon and colon cancer tissues

| | Normal | | | | | | | | | LN | | | Liver | | |
|---|---|---|---|---|---|---|---|---|---|---|---|---|---|---|---|
| | Colon | | | Adenomas | | | Adenocarcinomas | | | Metastases | | | metastases | | |
| | H | M | L | H | M | L | H | M | L | H | M | L | H | M | L |
| *Fas protein | 100 | 0 | 0 | 64 | 36 | 0 | 36 | 29 | 36 | 40 | 20 | 40 | 0 | 14 | 86 |
| level (%) | 5/5 | 0/5 | 0/5 | 9/14 | 5/14 | 0/1 | 5/14 | 4/14 | 5/14 | 2/5 | 1/5 | 2/5 | 0/7 | 1/7 | 6/7 |

*H: high, M: medium L: low to undetectable. The Fas protein level in normal human colon tissues is set as high and used as reference for scoring Fas protein level in the tumor tissues. The number under the percentage indicates number of specimens in that category vs total number of specimens.

### Example 2: Fas Receptor is Expressed on Human Colon Carcinoma Cell Surface

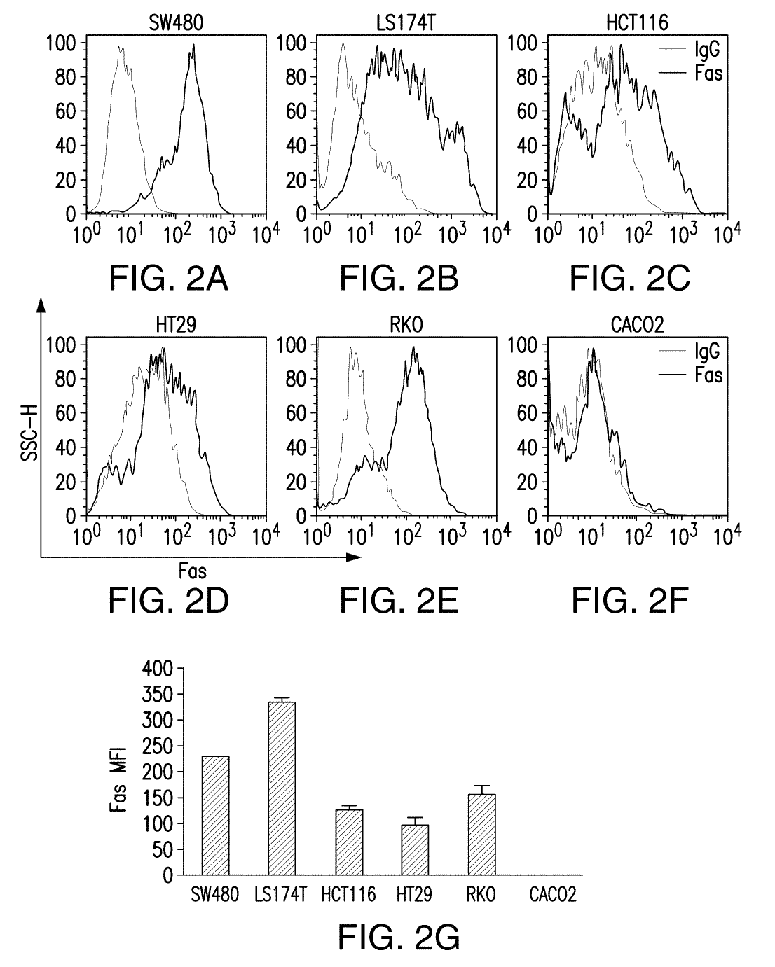

The above observations suggest that as the cancer progresses to advanced stages, colon carcinoma cells may progressively down-regulate Fas expression to decrease cell sensitivity to FasL. It is the Fas receptor expressed on the tumor cell surface that mediates FasL-induced apoptosis. Next, Fas protein levels on human colon carcinoma cell surfaces was analyzed. Among the six human colon carcinoma cell lines examined, Fas receptor levels are still high in three cell lines (SW480, LS174T and RKO), medium in two cell lines (HCT116 and HT29) and undetectable in CACO2 cells (FIG. 2A). These observations indicate that Fas receptor is expressed in the majority of human carcinoma cell lines.

### Example 3: Fas Receptor Level is not Correlated with Human Carcinoma Cell Sensitivity to FasL-Induced Apoptosis

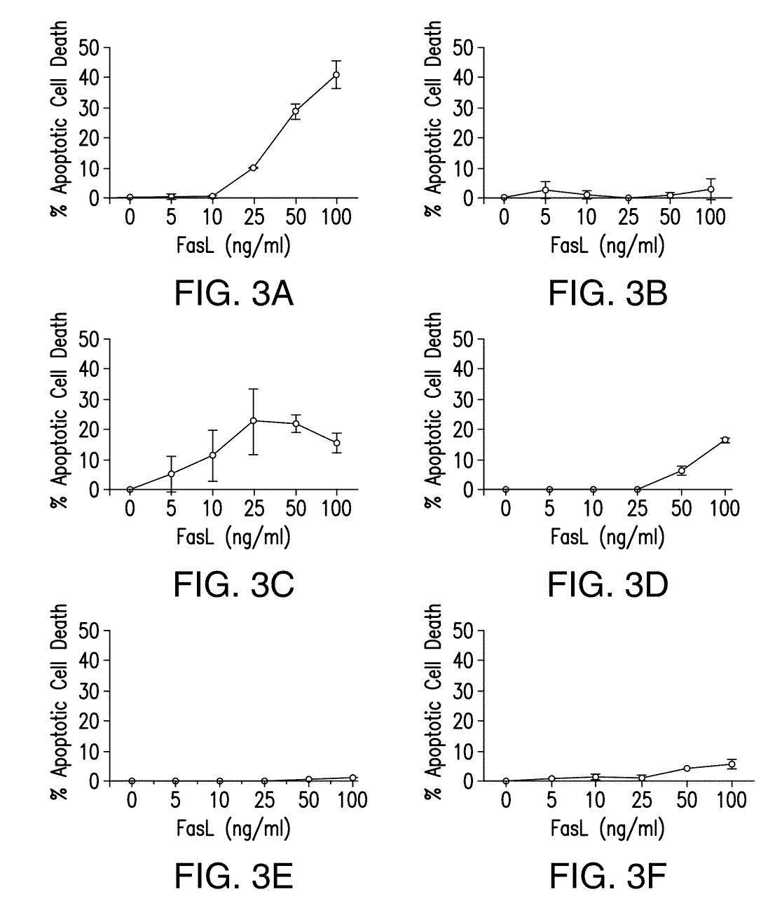

Fas receptor is expressed on the majority of human colon carcinoma cell line surfaces (FIGS. 2A-2G). To determine whether the Fas receptor level is associated with sensitivity of these human colon carcinoma cells to FasL-induced apoptosis, human colon carcinoma cells were treated with various doses of FasL and analyzed for apoptotic cell death. SW480 cells express high levels of Fas receptor and are sensitive to FasL-induced apoptosis (FIG. 3A). HCT116 cells express medium levels of Fas receptor and are as sensitive to FasL-induced apoptosis as SW480 cells (FIG. 3C). However, LS174T cells exhibit the highest Fas receptor levels (FIG. 3 among the six cell lines, but is less sensitive to FasL-induced apoptosis (FIG. 3D) as compared to SW480 and HCT116. RKO and HT29 cells express high to medium levels of Fas receptor and are not sensitive to FasL-induced apoptosis (FIGS. 3E and 3F). These observations thus indicate that the majority of human colon carcinoma cells have detectable Fas receptor on their surface, and Fas⁺ human colon carcinoma cells are not necessarily sensitive to FasL-induced apoptosis.

### Example 4: Development of Ceramide Analogs for Sensitization of Fas-Mediated Apoptosis

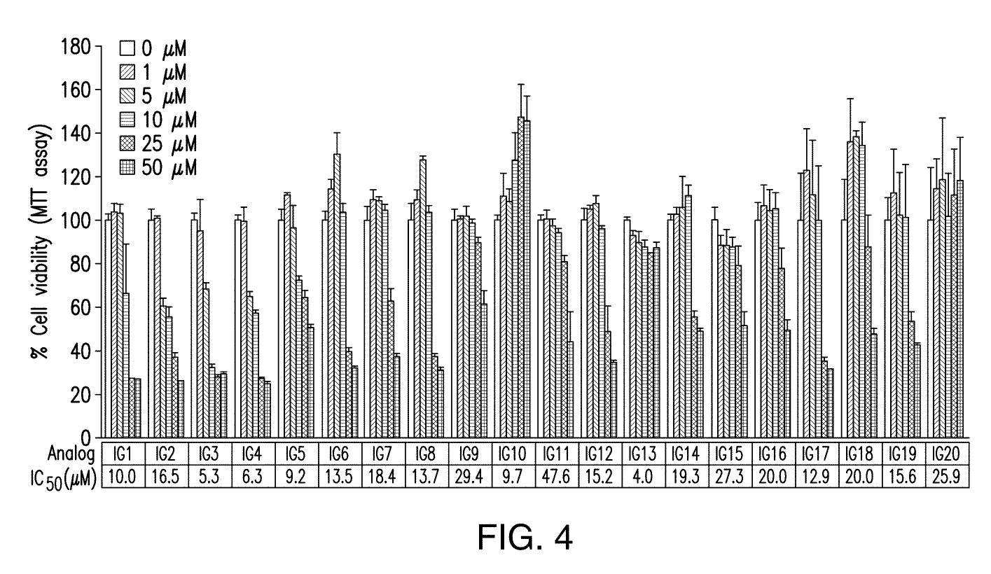

The structures and functions of ceramide analogs Cheng, J. C. et al. Radiation-induced acid ceramidase confers prostate cancer resistance and tumor relapse. J Clin Invest 123, 4344-4358, doi:10.1172/JCI6479164791 [pii] (2013); and Paschall, A. V. et al. Ceramide targets xIAP and cIAP1 to sensitize metastatic colon and breast cancer cells to apoptosis induction to suppress tumor progression. BMC Cancer 14, 24, doi:10.1186/1471-2407-14-241471-2407-14-24 [pii] (2014) were analyzed and synthesized twenty ceramide analogs to be developed into Fas sensitizers (Supplemental Table 1). The cytotoxicity of these twenty ceramide analogs was tested first using SW480 cells. These ceramide analogs have an IC50 ranging from about 5 to 50 μM (FIG. 4). Two of the analogs (IG10 and IG20) exhibited no cytotoxicity at the doses tested (FIG. 4).

### Example 5: Ceramide Analogs Sensitize Human Colon Carcinoma Cells to FasL-Induced Apoptosis

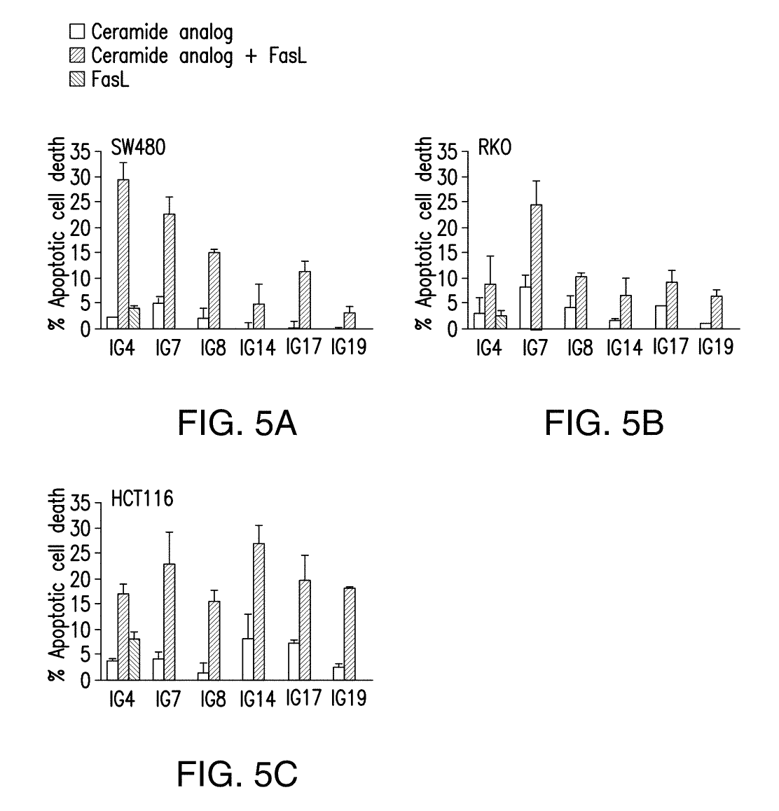

Next, the efficacy of the ceramide analogs was tested at their sublethal doses in enhancement of FasL-induced apoptosis using SW480, RKO and HCT116 cells. Tumor cells were treated with a sublethal dose of each of these 20 ceramide analogs along or ceramide analog plus FasL, and analyzed for apoptosis. Among the twenty ceramide analogs, six analogs (IG4, IG7, IG8, IG14, IG17 and IG19) exhibited significant efficacy in increasing the three human colon carcinoma cells to FasL-induced apoptosis (FIGS. 5A to 5C). Therefore, six novel ceramide analogs were identified that can effectively enhance the efficacy of FasL-induced apoptosis in human colon carcinoma cells.

### Example 6: Ceramide Analogs Increase FasL-Induced Caspase 8 Activation

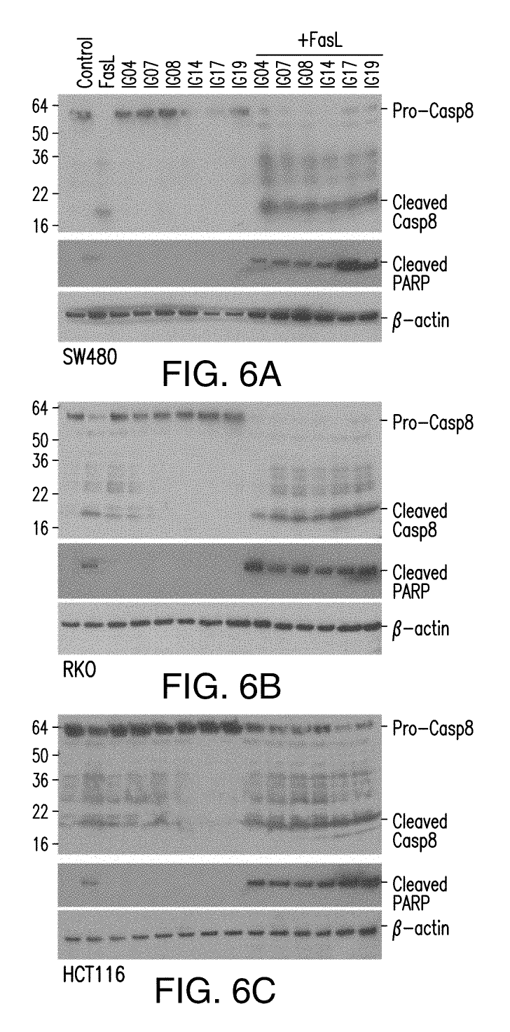

FasL binding to the Fas receptor induces DISC formation and subsequent caspase 8 activation that initiates the Fas-mediated apoptosis Kaufmann, T., Strasser, A. & Jost, P. J. Fas death receptor signalling: roles of Bid and XIAP. Cell Death Differ 19, 42-50, doi:cdd2011121 [pii]10.1038/cdd.2011.121 (2012). The hypothesis that these ceramide analogs modulate caspase 8 activation to increase human colon carcinoma cell sensitivity to FasL-induced apoptosis was. Tumor cells were treated with either FasL, ceramide analogs, or ceramide analogs plus FasL and analyzed for caspase 8 activation. Western blotting analysis indicates that FasL induces caspase 8 activation as evidenced by degradation of procaspase 8 and generation of cleaved caspase 8 in SW480 (FIG. 6A), RKO (FIG. 6B) and HCT116 (FIG. 6C) cells. None of the six ceramide analogs at their sublethal doses induces caspase 8 activation. However, combination of ceramide analog with FasL increased procaspase 8 degradation and generation of active caspase 8 in all three human colon carcinoma cell lines tested (FIG. 6A-6C). Furthermore, the cleavage of PARP, a biochemical indicator of apoptosis, was also enhanced by all 6 ceramide analogs (FIG. 6A-6C).

### Example 7: Ceramide Analogs Effectively Enhance Human Colon Carcinoma Cell Lysis Through FasL of Tumor-Specific CTLs

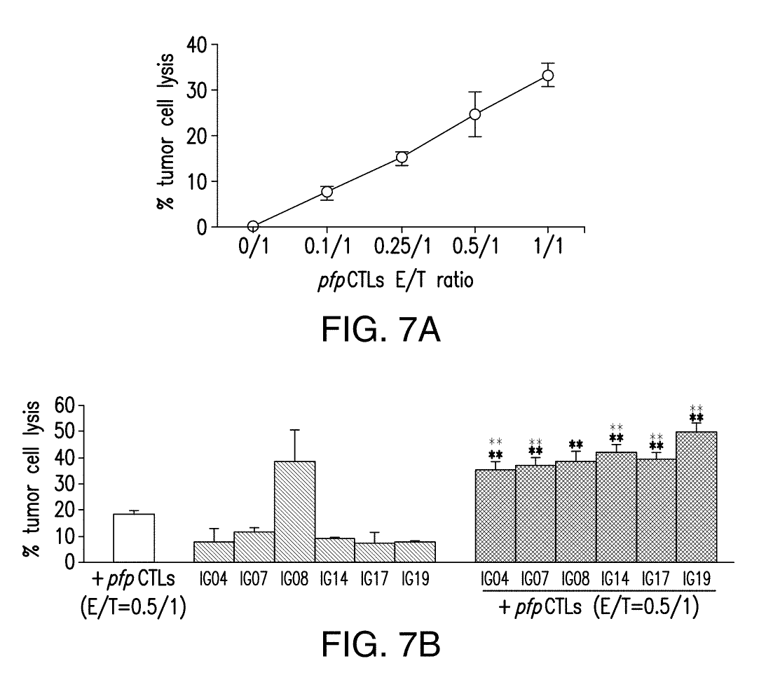

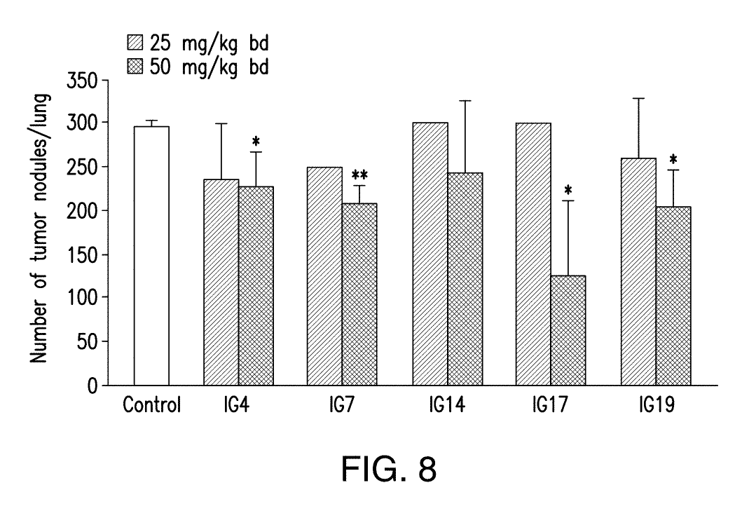

FasL of CTLs plays an essential role in host cancer immunosurveillance to suppress spontaneous cancer development Afshar-Sterle, S. et al. Fas ligand-mediated immune surveillance by T cells is essential for the control of spontaneous B cell lymphomas. Nat Med 20, 283-290, doi:10.1038/nm.3442 nm.3442 [pii] (2014); Caldwell, S. A., Ryan, M. H., McDuffie, E. & Abrams, S. I. The Fas/Fas ligand pathway is important for optimal tumor regression in a mouse model of CTL adoptive immunotherapy of experimental CMS4 lung metastases. J Immunol 171, 2402-2412 (2003); and Peyvandi, S. et al. Fas Ligand Deficiency Impairs Tumor Immunity by Promoting an Accumulation of Monocytic Myeloid-Derived Suppressor Cells. Cancer Res 75, 4292-4301, doi:10.1158/0008-5472.CAN-14-18480008-5472.CAN-14-1848 [pii] (2015). To determine whether the observation that these six ceramide analogs can sensitize FasL-induced apoptosis can be extended to CTL-mediated tumor lysis, a proof of principle study was performed. A perforin-deficient CTL line (pfpCTL) that recognizes mouse colon carcinoma cell line CT26 was used to determine whether these six ceramide analogs are effective in sensitizing CT26 tumor cells of FasL-mediated cytotoxicity of tumor-specific pfpCTLs. As expected, pfpCTLs kills CT26 cells in a dose-dependent manner (FIG. 7A). Addition of sublethal doses of ceramide analogs significantly increased the efficacy of pfpCTL-mediated lysis of CT26 tumor cells (FIG. 7B). One of the ceramide analogs, IG8, exhibits high cytotoxicity to CT26 tumor cells. The other five ceramide analogs exhibited low cytotoxicity at the dose used but showed dramatic efficacy in enhancement of the tumor-specific CTL activity in lysis of CT26 tumor cells. Taken together, five ceramide analogs were developed that exhibit high efficacy as adjunct agents in enhancement of the FasL-mediated effector mechanism of tumor-specific CTLs.

### Example 8: Synthesis of Ceramide Analogs

One objective was to synthesize ceramide analogs or N-CDase (ASAH2) inhibitors using L-threonine as starting material. The scheme for synthesizing ceramide analogs is as follows:

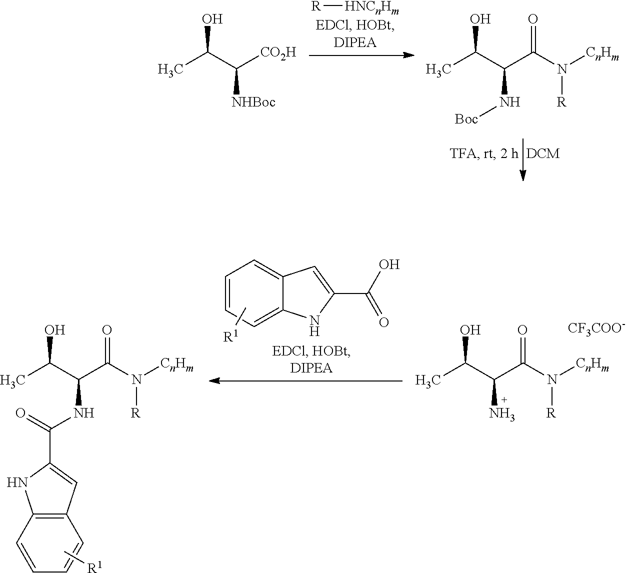

A library of ceramide analogs based on structure of ceramide was synthesized using this scheme. Synthesis of ceramide analogs that contain unsaturated moieties in the aliphatic tail and the nitro group substituent in the aromatic ring. IG4, IG7, IG8, IG14, IG17, and IG9 were the novel ceramide analogs which exhibited potent activity in human colon carcinoma.
These compounds were effective in activating tumor specific CTLs to induce cell apoptosis.
Activated CTLs induced Fas signaling pathway, which is seen by activation of caspase 8.

### Synthesis of Boc-Thr-Carboxamides:

To a solution of Boc-Thr-OH (1.0 eq) and amine (1.0 eq) in DMF hydroxybenzotriazole (HOBt) (1.2 eq) was added, EDCI and diisopropylethylamine (DIPEA) (2.5 eq). The mixture was stirred for 24 h at r.t. and subsequently diluted with five times its volume of ethyl acetate, washed twice with 2 M HCl, and two times with saturated NaHCO₃ solution, and brine. After drying over MgSO₄, solvent was evaporated.

### Deprotection of Boc-Thr-Carboxamides:

To a solution of Boc-Thr-carboxamide in DCM trifluoroacetic acid (TFA) was added, and the mixture was stirred for 30 min. After the reaction was completed (followed by TLC), reaction mixture was washed with ice-cold sodium hydroxide solution (10%) and water. After drying over MgSO₄, solvent was evaporated.

### Synthesis of Indolecarboxylate-Thr-Carboxamides:

To a solution of Boc-Thr-carboxamide (1.0 eq) and amine (1.0 eq) in DMF hydroxybenzotriazole (HOBt) (1.2 eq) was added followed by the addition of EDCI (1.0 eq.) and DIPEA (2.5 eq). The mixture was stirred for 24 h and subsequently diluted with five times its volume of ethyl acetate, washed twice with 2 M HCl, and two times with saturated NaHCO₃ solution, and brine. After drying over MgSO4, solvent was evaporated.

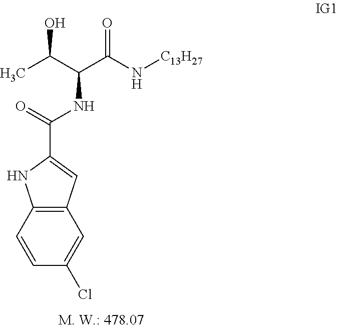

### 5-Chloro-N-((2S,3R)-3-hydroxy-1-oxo-1-(tridecylamino)butan-2-yl)-1H-indole-2-carboxamide

yellow solid (86% yield) mp: 126-129° C.; ¹H NMR (300 MHz, CDCl₃) δ 9.41 (s, 1H), 7.66 (d, J=2.0 Hz, 1H), 7.39-7.26 (m, 3H), 6.98 (dd, J=3.0 Hz, 1H), 6.77 (t, J=6.0 Hz, 1H), 4.51 (dt, J=7.8, 2.4 Hz, 2H), 3.26 (m, 2H), 1.54-1.44 (m, 2H), 1.26-1.22 (m, 24H), 0.96-0.85 (t, J=3.0 Hz, 3H); ¹³C NMR (75 MHz, CDCl₃) δ 170.7, 162.1, 134.8, 130.9, 128.5, 126.6, 125.5, 121.4, 113.0, 103.2, 66.6, 56.9, 39.7, 31.9, 29.6, 29.6, 29.5, 29.5, 29.5, 29.4, 29.3, 29.2, 26.9, 22.7, 18.3, 14.1.

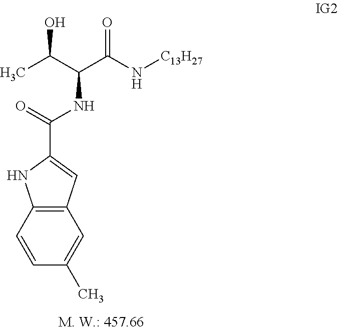

### N-((2S,3R)-3-Hydroxy-1-oxo-1-(tridecylamino)butan-2-yl)-5-methyl-1H-indole-2-carboxamide

mp: 127-129° C.; ¹H NMR (300 MHz, CDCl₃) δ 9.43 (s, 1H), 9.45 (d, J=3.0, 1H), 7.35-7.24 (m, 2H), 7.15 (d, J=9.0 Hz, 1H), 6.97 (s, 1H), 6.79 (t, J=6.0 Hz, 1H), 4.55-4.49 (m, 2H), 3.33-3.21 (m, 2H), 2.46 (s, 3H), 1.51-1.48 (m, 2H), 1.35-1.19 (m, 14H), 0.90 (t, J=6.0, 3H); ¹³C NMR (75 MHz, CDCl₃) δ 171.0, 162.6, 134.9, 130.3, 129.5, 127.9, 127.0, 121.5, 111.5, 103.2, 66.5, 56.6, 39.6, 31.9, 29.6, 29.6, 29.6, 29.5, 29.5, 29.4, 29.3, 29.2, 26.8, 22.7, 21.4, 18.2, 14.1.

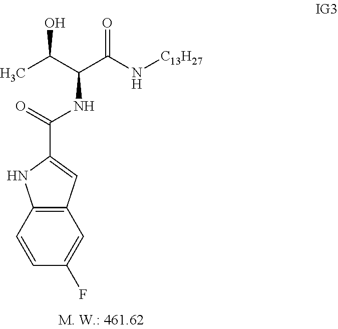

### 5-Fluoro-N-((2S,3R)-3-hydroxy-1-oxo-1-(tridecylamino)butan-2-yl)-1H-indole-2-carboxamide

mp: 141-143° C.; ¹H NMR (300 MHz, CDCl₃) δ 9.27 (s, 1H), 7.40-7.29 (m, 2H), 7.27-7.06 (m, 2H), 7.00 (d, J=3.0 Hz, 1H), 6.79-6.77 (m, 1H), 4.56-4.48 (m, 2H), 3.32-3.24 (m, 2H), 1.54-1.44 (m, 2H), 1.30-1.22 (m, 24H), 0.90 (t, J=6.0 Hz, 3H); ¹³C NMR (75 MHz, CDCl₃) δ 170.8, 162.2, 159.8, 156.7, 133.1, 131.1, 127.8, 113.9, 112.7, 106.7, 103.6, 66.9, 56.7, 39.7, 31.9, 29.6, 29.6, 29.5, 29.5, 29.3, 29.3, 29.2, 26.8, 22.7, 18.3, 14.1.

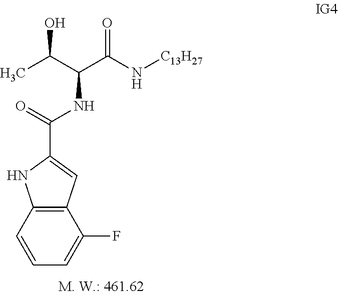

### 4-Fluoro-N-((2S,3R)-3-hydroxy-1-oxo-1-(tridecylamino)butan-2-yl)-1H-indole-2-carboxamide

mp: 81-86° C.; ¹H NMR (300 MHz, CDCl₃) δ 9.85 (s, 1H), 8.03 (s, 1H), 7.53 (dd, J=10.3, 8.1 Hz, 1H), 7.23-7.08 (m, 2H), 6.96-6.65 (m, 2H), 4.72-4.31 (m, 2H), 3.26 (dt, J=13.0, 7.1 Hz, 2H), 2.97 (d, J=0.5 Hz, 3H), 1.40-1.05 (m, 23H), 1.05-0.64 (m, 3H); ¹³C NMR (75 MHz, CDCl₃) δ 170.7, 162.1, 138.9, 129.8, 125.5, 117.2, 107.9, 105.3, 105.0, 100.0, 66.6, 57.1, 53.7, 39.7, 31.9, 29.6, 29.6, 29.5, 29.5, 29.3, 29.3, 29.2, 26.9, 22.7, 18.6, 14.1.

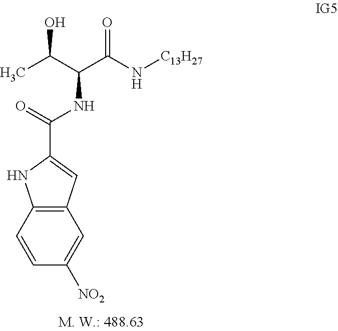

### N-((2S,3R)-3-Hydroxy-1-oxo-1-(tridecylamino)butan-2-yl)-5-nitro-1H-indole-2-carboxamide

mp: 184-192° C.; ¹H NMR (300 MHz, CDCl₃) δ 9.85-9.75 (m, 2H), 8.69-8.67 (m, 1H), 8.25-8.19 (m, 1H), 7.63 (d, J=6.0 Hz, 1H), 7.24 (s, 1H), 7.06-6.93 (m, 1H), 3.52 (d, J=6.9 Hz, 1H), 3.40-3.12 (m, 2H), 1.58-1.20 (m, 27H), 0.96-0.83 (t, J=6.0 Hz, 3H).

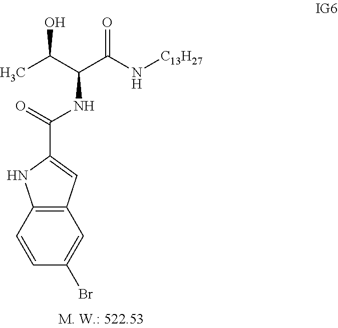

### 5-Bromo-N-((2S,3R)-3-hydroxy-1-oxo-1-(tridecylamino)butan-2-yl)-1H-indole-2-carboxamide

mp: 127-134° C.; ¹H NMR (300 MHz, CDCl₃) δ 9.50 (s, 1H), 7.80 (s, 1H), 7.47-7.29 (m, 2H), 6.99 (s, 1H), 6.79 (t, J=5.8 Hz, 1H), 4.63-4.44 (m, 2H), 3.36-3.18 (m, 2H), 1.79 (br. s, 2H), 1.53-1.32 (m, 2H), 1.27-1.18 (m, 23H), 0.89 (t, J=6.0, 3H); ¹³C NMR (75 MHz, CDCl₃) δ 170.7, 162.1, 135.1, 130.7, 129.2, 128.0, 124.6, 114.0, 113.4, 103.1, 66.6, 56.9, 39.7, 31.9, 29.6, 29.6, 29.6, 29.5, 29.5, 29.4, 29.3, 29.2, 26.9, 22.7, 18.3, 14.1.

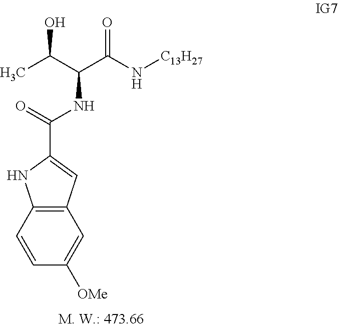

### N-((2S,3R)-3-Hydroxy-1-oxo-1-(tridecylamino)butan-2-yl)-5-methoxy-1H-indole-2-carboxamide

mp: 103-106° C.; ¹H NMR (300 MHz, CDCl₃) δ 9.91 (s, 1H), 7.56 (d, J=7.6 Hz, 1H), 7.31 (dt, J=8.9, 0.7 Hz, 1H), 7.04-6.98 (m, 2H), 6.93 (dd, J=8.9, 2.5 Hz, 1H), 6.75 (s, 1H), 4.55 (dd, J=7.7, 2.9 Hz, 1H), 4.42 (dd, J=6.5, 2.9 Hz, 1H), 3.83 (s, 3H), 3.22 (ddq, J=20.3, 13.2, 7.1, 6.7 Hz, 2H), 1.35-1.12 (m, 26H), 0.89 (t, J=6.0 Hz, 3H); ¹³C NMR (75 MHz, CDCl₃) δ 171.0, 162.5, 154.9, 131.8, 129.9, 128.0, 116.6, 112.7, 103.3, 102.4, 66.46, 56.6, 55.7, 39.6, 31.9, 29.6, 29.6, 29.6, 29.5, 29.5, 29.3, 29.3, 29.2, 26.5, 22.7, 18.2, 14.1.

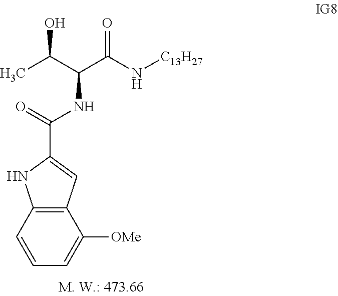

### N-((2S,3R)-3-Hydroxy-1-oxo-1-(tridecylamino)butan-2-yl)-4-methoxy-1H-indole-2-carboxamide

mp: 100-101° C.; ¹H NMR (300 MHz, CDCl₃) δ 9.33 (s, 1H), 8.04 (s, 1H), 7.29-7.16 (m, 2H), 7.04 (dt, J=8.3, 0.8 Hz, 1H), 6.82 (t, J=5.8 Hz, 1H), 6.63 (d, J=6.0 Hz, 1H), 4.65-4.44 (m, 2H), 3.97 (s, 3H), 3.26 (qd, J=7.0, 3.2 Hz, 2H), 2.98 (s, 3H), 2.90 (s, 3H), 1.37-1.16 (m, 20H), 0.89 (t, J=6.0 Hz, 3H); ¹³C NMR (75 MHz, CDCl₃) δ 170.9, 162.6, 154.3, 138.1, 128.4, 125.9, 119.0, 105.0, 101.7, 99.8, 66.7, 57.1, 55.3, 39.7, 31.9, 29.7, 29.6, 29.6, 29.6, 29.5, 29.4, 29.3, 29.2, 26.9, 22.7, 18.3, 14.1.

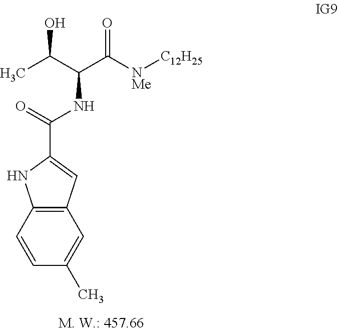

### N-((2S,3R)-1-(Dodecyl(methyl)amino)-3-hydroxy-1-oxobutan-2-yl)-5-methyl-1H-indole-2-carboxamide

oil, ¹H NMR (300 MHz, CDCl₃) δ 9.62 (d, J=18.1 Hz, 1H), 7.50-7.31 (m, 2H), 7.11 (dd, J=8.4, 1.5 Hz, 1H), 7.06-6.94 (m, 1H), 5.06 (dt, J=9.2, 2.3 Hz, 1H), 4.24 (qt, J=5.4, 2.4 Hz, 1H), 3.65 (ddd, J=15.1, 8.8, 6.7 Hz, 1H), 3.57-3.24 (m, 2H), 3.18 (s, 3H), 2.97 (s, 3H), 2.45 (s, 3H), 1.57-1.52 (m, 2H), 1.43-1.03 (m, 19H), 0.89 (t, J=6.0 Hz, 3H); ¹³C NMR (75 MHz, CDCl₃) δ 172.0, 171.6, 134.9, 130.0, 129.7, 127.9, 126.7, 121.4, 111.5, 67.9, 52.6, 51.8, 50.2, 48.4, 35.6, 33.8, 31.9, 29.6, 29.5, 29.3, 26.8, 26.6, 22.7, 21.4, 19.0, 19.0, 14.1.

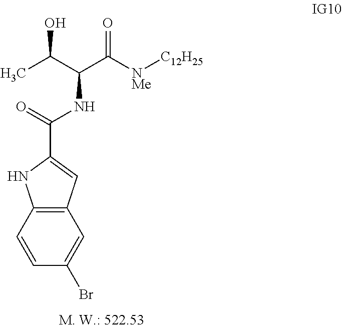

### 5-Bromo-N-((2S,3R)-1-(dodecyl(methyl)amino)-3-hydroxy-1-oxobutan-2-yl)-1H-indole-2-carboxamide

oil, ¹H NMR (300 MHz, CDCl₃) δ 9.71 (br. s, 1H), 7.78 (br. s, 1H), 7.46-7.21 (m, 2H), 6.99 (s, 1H), 5.52 (t, J=8.0 Hz, 1H), 5.07 (ddd, J=9.3, 4.1, 2.2 Hz, 1H), 4.47 (d, J=9.8 Hz, 1H), 4.25 (ddd, J=6.7, 4.7, 2.1 Hz, 1H), 4.10 (t, J=6.1 Hz, 1H), 3.78-3.27 (m, 1H), 3.18 (s, 3H), 2.97 (s, 3H), 1.38-0.99 (m, 20H), 0.99-0.79 (t, J=6.0 Hz, 3H); ¹³C NMR (75 MHz, CDCl₃) δ 171.7, 161.3, 135.0, 130.9, 127.7, 124.5, 113.9, 111.7, 103.1, 100.7, 68.1, 52.7, 50.2, 48.4, 35.6, 33.9, 31.9, 29.6, 29.5, 29.4, 29.3, 28.3, 26.8, 22.7, 19.1, 14.1.

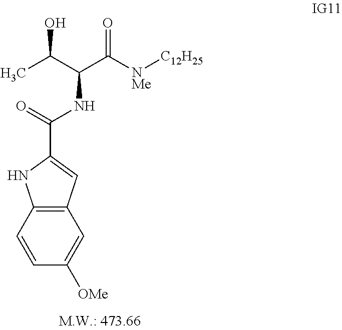

### N-((2S,3R)-1-(Dodecyl(methyl)amino)-3-hydroxy-1-oxobutan-2-yl)-5-methoxy-1H-indole-2-carboxamide

oil, ¹H NMR (300 MHz, CDCl₃) δ 9.30 (d, J=11.2 Hz, 1H), 7.51-6.81 (m, 5H), 5.05 (ddd, J=9.5, 4.9, 1.9 Hz, 1H), 4.24 (t, J=6.1 Hz, 1H), 3.78-3.58 (m, 1H), 3.56-3.25 (m, 1H), 3.19 (s, 3H), 2.98 (s, 3H), 1.75 (s, 2H), 1.58-1.54 (m, 1H), 1.28-1.22 (m, J=6.9, 4.3 Hz, 21H), 0.89 (t, J=6.8 Hz, 3H); ¹³C NMR (75 MHz, CDCl₃) δ 171.9, 161.6, 154.7, 131.9, 130.2, 128.0, 116.2, 112.7, 103.4, 102.4, 68.0, 55.7, 52.5, 51.7, 50.1, 48.4, 35.6, 33.8, 31.9, 29.6, 29.4, 29.3, 28.8, 26.8, 22.7, 19.0, 14.1.

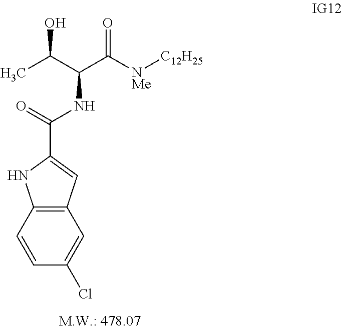

### 5-Chloro-N-((2S,3R)-1-(dodecyl(methyl)amino)-3-hydroxy-1-oxobutan-2-yl)-1H-indole-2-carboxamide

oil, ¹H NMR (300 MHz, CDCl₃) δ 9.54 (d, J=5.0 Hz, 1H), 7.63 (t, J=2.3 Hz, 1H), 7.56-7.19 (m, 3H), 6.97 (s, 1H), 5.06 (ddd, J=9.3, 4.3, 2.0 Hz, 1H), 4.24 (ddd, J=6.6, 4.8, 2.0 Hz, 1H), 3.82-3.55 (m, 1H), 3.54-3.32 (m, 1H), 3.19 (s, 3H), 2.99 (s, 3H), 1.68-1.42 (m, 2H), 1.43-1.05 (m, 19H), 0.89 (t, J=6.8 Hz, 3H); ¹³C NMR (75 MHz, CDCl₃) δ 171.8, 161.2, 134.7, 131.1, 128.6, 126.4, 125.3, 121.4, 112.9, 103.1, 67.9, 52.5, 51.8, 50.2, 48.4, 35.6, 33.9, 31.9, 29.6, 29.5, 29.3, 28.8, 27.0, 22.7, 19.1, 14.1.

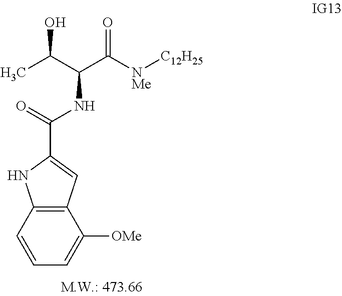

### N-((2S,3R)-1-(Dodecyl(methyl)amino)-3-hydroxy-1-oxobutan-2-yl)-4-methoxy-1H-indole-2-carboxamide

oil, ¹H NMR (300 MHz, CDCl₃) δ 9.40 (d, J=14.4 Hz, 1H), 7.26-7.12 (m, 3H), 7.03 (d, J=8.3, 0.9 Hz, 1H), 6.52 (d, J=7.7 Hz, 1H), 5.03 (ddd, J=9.5, 4.8, 1.9 Hz, 1H), 4.62-4.04 (m, 1H), 3.96 (s, 3H), 3.49-3.39 (m, 1H), 3.15 (d, J=18.0 Hz, 2H), 2.96 (d, J=8.1 Hz, 2H), 1.38-1.08 (m, 24H), 0.89 (t, J=6.8 Hz, 3H); ¹³C NMR (75 MHz, CDCl₃) δ 171.9, 161.6, 154.4, 137.9, 128.4, 125.7, 119.1, 104.8, 101.5, 68.0, 55.3, 52.4, 51.6, 50.1, 48.4, 35.6, 33.8, 31.9, 29.6, 29.6, 29.4, 29.3, 28.3, 26.9, 22.7, 18.9, 14.1.

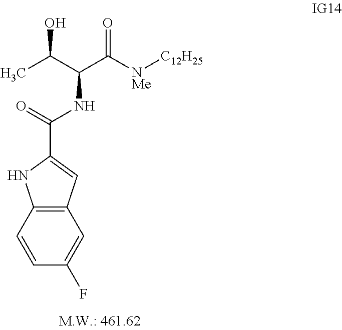

### N-((2S,3R)-1-(Dodecyl(methyl)amino)-3-hydroxy-1-oxobutan-2-yl)-5-fluoro-1H-indole-2-carboxamide

oil, ¹H NMR (300 MHz, CDCl₃) δ 9.55 (d, J=8.2 Hz, 1H), 7.60-6.76 (m, 5H), 5.07 (ddd, J=9.4, 4.6, 2.0 Hz, 1H), 4.25 (ddd, J=6.5, 4.9, 2.0 Hz, 1H), 3.66 (dt, J=15.1, 7.4 Hz, 1H), 3.62-3.28 (m, 1H), 3.19 (s, 3H), 1.68-1.47 (m, 2H), 1.45-1.09 (m, 22H), 0.89 (t, J=6.8 Hz, 3H); ¹³C NMR (75 MHz, CDCl₃) δ 171.8, 161.4, 133.1, 127.9, 113.5, 112.8, 112.6, 106.6, 103.7, 68.1, 67.9, 52.7, 51.9, 50.2, 48.5, 35.6, 33.9, 31.9, 29.6, 29.5, 29.3, 27.0, 26.8, 22.7, 19.1, 14.1.

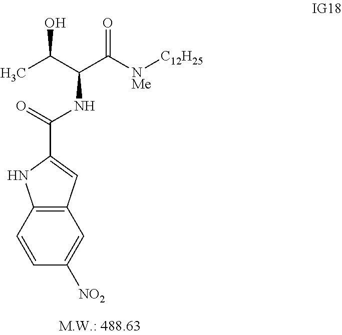

### N-((2S,3R)-1-(Dodecyl(methyl)amino)-3-hydroxy-1-oxobutan-2-yl)-5-nitro-1H-indole-2-carboxamide

mp: 114-130° C.; ¹H NMR (300 MHz, CDCl₃) δ 10.16 (d, J=12.4 Hz, 1H), 8.60 (dd, J=5.3, 2.2 Hz, 1H), 8.17 (ddd, J=9.1, 3.7, 2.2 Hz, 1H), 7.78-7.56 (m, 1H), 7.60-7.39 (m, 2H), 7.20 (ddd, J=6.1, 2.1, 0.9 Hz, 1H), 5.11 (ddd, J=8.9, 6.2, 2.3 Hz, 1H), 4.71-3.99 (m, 2H), 3.80-3.27 (m, 2H), 3.46 (s, 3H), 1.75-1.47 (m, 2H), 1.36-1.12 (m, 20H), 89 (t, J=6.0 Hz, 3H); ¹³C NMR (75 MHz, CDCl₃) δ 171.1, 161.0, 142.4, 139.2, 133.3, 126.7, 119.4, 112.1, 106.1, 68.2, 53.2, 52.4, 50.2, 48.6, 35.7, 33.9, 31.9, 29.6, 29.5, 29.4, 28.8, 28.3, 27.0, 26.9, 22.6, 14.1.

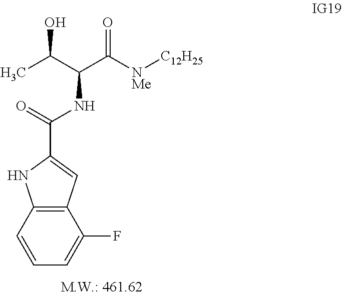

### N-((2S,3R)-1-(Dodecyl(methyl)amino)-3-hydroxy-1-oxobutan-2-yl)-4-fluoro-1H-indole-2-carboxamide

oil, ¹H NMR (300 MHz, CDCl₃) δ 9.70 (d, J=12.7 Hz, 1H), 7.43 (dd, J=18.8, 9.1 Hz, 1H), 7.28-7.07 (m, 3H), 6.87-6.70 (m, 1H), 5.07 (ddd, J=9.3, 3.9, 2.0 Hz, 1H), 4.33-4.19 (m, 1H), 3.75-3.25 (m, 2H), 3.15 (d, J=19.1 Hz, 2H), 2.97 (d, J=10.9 Hz, 2H), 1.69-1.47 (m, 3H), 1.41-1.09 (m, 20H), 0.89 (t, J=6.8 Hz, 3H); ¹³C NMR (75 MHz, CDCl₃) δ 171.7, 161.5, 158.68, 138.8, 130.0, 125.2, 117.5, 107.9, 105.2, 99.8, 68.1, 52.7, 51.9, 50.2, 48.4, 35.6, 33.9, 31.9, 29.6, 29.4, 28.8, 28.3, 26.9, 22.7, 19.1, 14.1.

### N-((2S,3R)-3-hydroxy-1-(methyl(octadecyl)amino)-1-oxobutan-2-yl)-5H-[1,3]dioxolo[4,5-f]indole-6-carboxamide

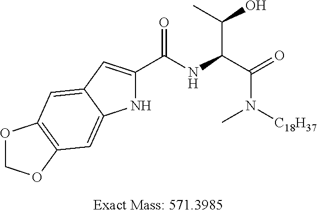

### N-((2S,3R)-1-(dodecyl(methyl)amino)-3-hydroxy-1-oxobutan-2-yl)-5H-[1,3]dioxolo[4,5-f]indole-6-carboxamide

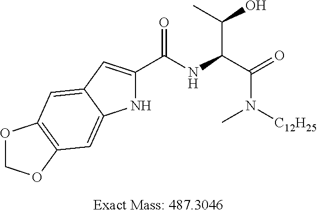

### N-((2S,3R)-3-hydroxy-1-(methyl(tridecyl)amino)-1-oxobutan-2-yl)-5H-[1,3]dioxolo[4,5-f]indole-6-carboxamide

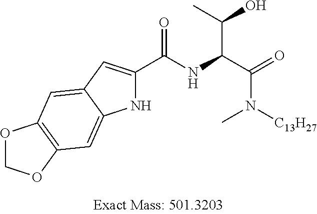

### N-((1S,2R)-1,3-dihydroxy-1-(4-nitrophenyl)propan-2-yl)oleamide IG17

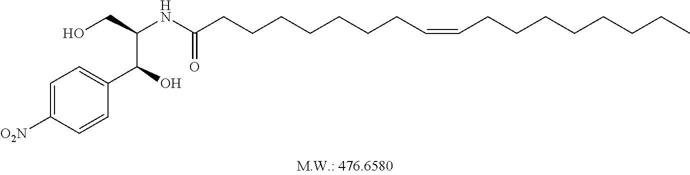 [SMILES: CCCCCCCC/C=C\CCCCCCCC(=O)N[C@H](CO)[C@@H](O)c1ccc([N+](=O)[O-])cc1]

HRIVIS (ESI) calcd for C₂₇H₄₄N₂O₅ [M+H]⁺ 476.3250, found 477.3313.

### (9Z,12Z)—N-((1S,2R)-1,3-dihydroxy-1-(4-nitrophenyl)propan-2-yl)octadeca-9,12-dienamide IG15

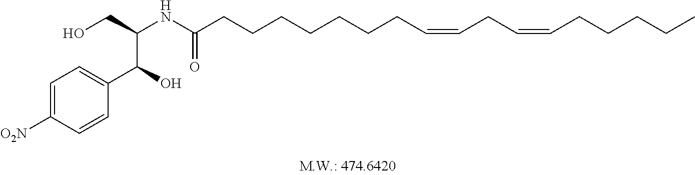 [SMILES: CCCCC/C=C\C/C=C\CCCCCCCC(=O)N[C@H](CO)[C@@H](O)c1ccc([N+](=O)[O-])cc1]

HRMS (ESI) calcd for C₂₇H₄₂N₂O₅ [M+H]⁺ 474.6420, found 475.3159.

### (E)-N-((1S,2R)-1,3-dihydroxy-1-(4-nitrophenyl)propan-2-yl)tetradec-9-enamide IG20

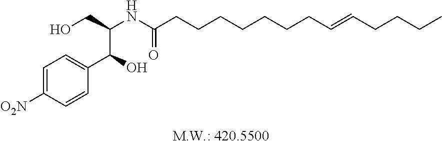 [SMILES: CCCC/C=C/CCCCCCCC(=O)N[C@H](CO)[C@@H](O)c1ccc([N+](=O)[O-])cc1]

HRMS (ESI) calcd for C₂₃H₃₆N₂O₅ [M+H]⁺ 420.2624, found 421.2706.

### (Z)—N-((1S,2R)-1,3-dihydroxy-1-(4-nitrophenyl)propan-2-yl)hexadec-9-enamide IG16

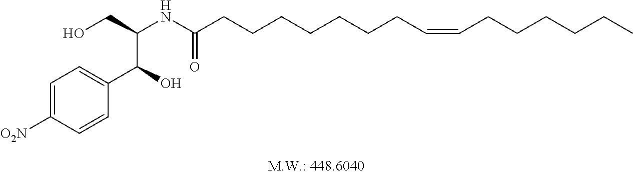 [SMILES: CCCCCC/C=C\CCCCCCCC(=O)N[C@H](CO)[C@@H](O)c1ccc([N+](=O)[O-])cc1]

HRMS (ESI) calcd for C₂₅H₄₀N₂O₅ [M+H]⁺ 448.2937, found 449.3002.

### Example 9: IG4, IG7, IG17 and IG19 Suppress the Established Colon Carcinoma Lung Metastasis In Vivo

### Materials and Methods

CT26 cells (2.5×10⁵ cell per mouse) were injected to BALB/c mice on subcutaneously. Tumor-bearing mice were treated with the 5 ceramide analogs (25 and 50 kg body weight) by intraperitoneal injection at days 8, 10 and 12 after tumor injection. Mice were sacrificed on day. Mouse lungs were inflated with ink and fixed. Top panel shows the tumor-bearing lungs. White dots are tumor nodules. Bottom panel: quantification of tumor nodule number in the lungs.

### Results

IG4, IG7, IG17 and IG19 suppress the established colon carcinoma lung metastasis in vivo.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention

1. A compound defined according to formula (I) as follows: 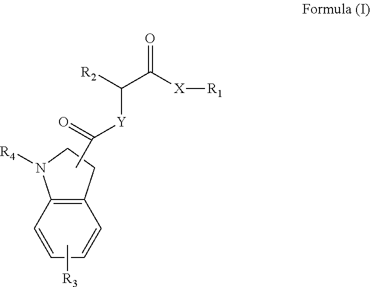
wherein X and Y are independently selected to be O, NH, S, or NR₅;
wherein R₁ is selected from C₁-C₃₀ linear, branched, or cyclic, optionally substituted alkyl, heteroalkyl, alkenyl, or alkynyl groups;
wherein R₂ is selected to be a hydrogen, C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₃ is selected from hydrogen, C₁-C₃₀ linear, branched, or cyclic, optionally substituted alkyl, aromatic, or heteroaromatic group or a halo substituent (i.e., F, Cl, Br, I), C₁-C₃₀ optionally substituted alkoxyl, or a nitro (—NO₂) group;
wherein R₄ is selected from hydrogen, C₁-C₃₀ linear or branched substituted or unsubstituted alkyl, alkenyl, or alkynyl groups; and
wherein R₅ is selected from C₁-C₃₀ linear or branched substituted or unsubstituted alkyl, alkenyl, or alkynyl groups.

2. The compound of claim 1, wherein substituent R₃ may independently substitute one or more positions of the benzene ring of the indole, as permitted by valency.

3. The compound of claim 1, wherein R₃ is selected to be a chloro, bromo, fluoro, methyl, nitro, or methoxy group.

4. The compound of claim 1, wherein X and Y are NH.

5. The compound of claim 1, wherein, Y is NR₅, and wherein R₅ is a methyl group.

6. The compound of claim 1, wherein R₁ is a C₁-C₃₀ alkyl group.

7. A compound defined according to formula (II) as follows: 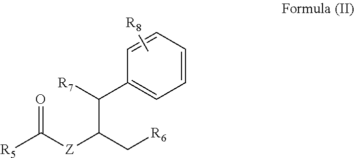
wherein Z selected to be O, NH, S, or NR₉;
wherein R₅ is selected from C₁-C₃₀ linear, branched, or cyclic, optionally substituted alkyl, heteroalkyl, alkenyl, or alkynyl groups;
wherein R₆ and R₇ are independently selected to be a hydrogen, hydroxyl, or C₁-C₃₀ linear, branched, or cyclic optionally substituted alkyl, alkenyl, or alkynyl groups or optionally substituted heterocylic groups; wherein the one or more substituents include, but are not limited to, selenyl (—SeH), thiol (—SH), amido (—C(O)NH₂), hydroxyl (—OH), amino, imidazolyl, guanidinyl, carboxyl (—C(O)OH), or carboxylate (—C(O)O⁻) groups; wherein R₈ is selected from hydrogen, C₁-C₃₀ linear, branched, or cyclic, optionally substituted alkyl, halo substituent (i.e., F, Cl, Br, I), C₁-C₃₀ optionally substituted alkoxyl, or nitro (—NO₂) groups; and wherein R₉ is selected from C₁-C₃₀ linear or branched substituted or unsubstituted alkyl, alkenyl, or alkynyl groups.

8. The compound of claim 7, wherein R₇ and R₈ are hydroxyl groups.

9. The compound of claim 7, wherein R₈ is a nitro, chloro, bromo, fluoro, methyl, or methoxy group.

10. The compound of claim 7, wherein substituent R₈ may independently substitute one or more positions of the phenyl group, as permitted by valency.

11. The compound of claim 7, wherein Z is NH.

12. The compound of claim 1, wherein R₅ is a C₁-C₃₀ alkyl group, or a C₅-C₂₀ alkyl, or a C₁₀-C₁₅ alkenyl group.

13.-19. (canceled)

20. A pharmaceutical composition comprising the compound of claim 7 and a pharmaceutically acceptable excipient.

21. A method for increasing cancer cell sensitivity to FasL-induced apoptosis comprising administering an effective amount of one or more ceramide analogs to cancer cells to enhance Fas oligomerization and to increase caspase-8 activity in the cancer cells.

22. The method of claim 21 wherein the ceramide analog is selected from the group consisting of 4-Fluoro-N-((2S,3R)-3-hydroxy-1-oxo-1-(tridecylamino)butan-2-yl)-1H-indole-2-carboxamide, N-((2S,3R)-3-Hydroxy-1-oxo-1-(tridecylamino)butan-2-yl)-5-methoxy-1H-indole-2-carboxamide, N-((2S,3R)-3-Hydroxy-1-oxo-1-(tridecylamino)butan-2-yl)-4-methoxy-1H-indole-2-carboxamide, N-((2S,3R)-1-(Dodecyl(methyl)amino)-3-hydroxy-1-oxobutan-2-yl)-5-fluoro-1H-indole-2-carboxamide, N-((2S,3R)-1-(Dodecyl(methyl)amino)-3-hydroxy-1-oxobutan-2-yl)-4-fluoro-1H-indole-2-carboxamide, and N-((1S,2R)-1,3-dihydroxy-1-(4-nitrophenyl)propan-2-yl)oleamide.

23. (canceled)

24. A method for increasing CTL-mediated and FasL-induced apoptosis of cancer cells in a subject comprising:
administering to a subject in need thereof an effective amount of a ceramide analog selected from the group consisting of 4-Fluoro-N-((2S,3R)-3-hydroxy-1-oxo-1-(tridecylamino)butan-2-yl)-1H-indole-2-carboxamide, N-((2S,3R)-3-Hydroxy-1-oxo-1-(tridecylamino)butan-2-yl)-5-methoxy-1H-indole-2-carboxamide, N-((2S,3R)-3-Hydroxy-1-oxo-1-(tridecylamino)butan-2-yl)-4-methoxy-1H-indole-2-carboxamide, N-((2S,3R)-1-(Dodecyl(methyl)amino)-3-hydroxy-1-oxobutan-2-yl)-5-fluoro-1H-indole-2-carboxamide, N-((2S,3R)-1-(Dodecyl(methyl)amino)-3-hydroxy-1-oxobutan-2-yl)-4-fluoro-1H-indole-2-carboxamide, and N-((1S,2R)-1,3-dihydroxy-1-(4-nitrophenyl)propan-2-yl)oleamide to increase cancer cell sensitivity to FasL-induced apoptosis.

25.-37. (canceled)
